(19) United States
(12) Patent Application Publication
(10) Pub. No.: US 2019/0298786 A1
(43) Pub. Date: Oct. 3, 2019

(54) EXTRACTS OF SAUDI ARABIAN HERBAL PLANTS, ANTI-CANCER METHOD USING SUCH EXTRACTS, AND CYTOLOGICAL PROFILING USING AUTOMATED HIGH-CONTENT IMAGING TECHNIQUE

(71) Applicant: King Abdullah University of Science and Technology, Thuwal (SA)

(72) Inventors: Dina Ahmed Hajjar, Thuwal (SA); Stephan Kremb, Abu Dhabi (AE); Christian Voolstra, Thuwal (SA); Timothy Ravasi, Thuwal (SA)

(21) Appl. No.: 16/335,602

(22) PCT Filed: Sep. 14, 2017
(86) PCT No.: PCT/IB2017/055579

§ 371 Date: Mar. 21, 2019

Related U.S. Application Data
(60) Provisional application No. 62/397,578, filed on Sep. 21, 2016.

(51) Int. Cl. A61K 36/14; A61K 36/31; A61K 36/42; A61P 35/00

(52) U.S. Cl. CPC A61K 36/14; A61P 35/00; A61K 36/42; A61K 36/31

(57) ABSTRACT

Cell-based phenotypic profiling and image based high-content screening are used to gain insight into the mode of action and potential cellular targets of plants historically used to determine anti-cancer activity of Saudi Arabian plants Juniperus phoenicea (Arar), Anastatica hierochuntica (Kaff Maryam), and Citrullus colocynthis (Hanzal). The cytological profiles of fractions taken from the plants were compared with a set of reference compounds with known modes of action. Cluster analyses of the cytological profiles were performed, which revealed detailed information on the modes of action of the tested compounds as potential topoisomerase inhibitors. Cytological profiles showed that some of these compounds inhibited cell proliferation causing cell cycle disruption.

### RELATED APPLICATIONS

This application claims the benefit of priority of U.S. provisional application Ser. No. 62/397,578, filed Sep. 21, 2016 of identical title, the entire contents of which application is incorporated by reference in its entirety herein.

### FIELD OF THE INVENTION

The presented invention relates to extracts of certain Saudi Arabian medicinal herbs which have been found to be topoisomerase inhibitors and thus useful in the treatment of cancerous disease states and conditions. Pharmaceutical compositions based upon these extracts represent an additional aspect of the invention. Methods of treating cancerous disease states and conditions through the inhibition of topoisomerase represent an additional aspect of the invention. The invention also relates to a method of determining potential usefulness of medicinal herbs in the treatment of cancer.

### BACKGROUND AND OVERVIEW OF THE INVENTION

Cancer is a leading cause of morbidity and mortality worldwide, exceeding the number of cases of illness and death due to HIV/AIDS, malaria, and tuberculosis (Aggarwal et al., 2009; Tavakoli et al., 2012). In 2012, 8.2 million cancer-related deaths were recorded, approximately 14 million more cases were diagnosed, and its incidence is predicted to increase to as many as 22 million cases per year over the next 20 years (Cancer, 2014). As the size of the population and the incidence of cancer increase, so will the economic burden on society. The pathology of cancer derives from cellular hyperproliferation that mediates cell differentiation, apoptosis, cell growth and invasion, and angiogenesis and metastasis (Kuete and Efferth, 2011; Tavakoli et al., 2012; Vorobiof and Abratt, 2007).

A significant development in anti-cancer drug discovery was the characterization of Topoisomerase II (topo II) as the primary target of the effect chemotherapeutic compounds etoposide and doxorubicin (Alfarhan et al., 1998; Nitiss, 2009). Topo II is a key enzyme that modulates DNA topology by transferring an intact DNA duplex through a DNA helix, which has been cut in an ATP hydrolysis reaction, topo II is an essential element of the chromosomal scaffold and its mode of action is necessary for chromosome disentanglement during cell division. Several key pathways, such as DNA replication and recombination are dependent on the DNA strand passage functions of topo II (Cragg and Newman, 2004; Jackson and Bartek, 2009; Liu, 1989; Nitiss, 2009; Pommier et al., 1985).

High-content screening (HCS) optimizes the discovery of biologically active small molecules and their subsequent development into therapeutic compounds by the use of automated microscopy in conjunction with image analysis. A high-throughput platform based on imageprocessing software that reviews images from automated fluorescence microscopy [5±10], HCS enhances phenotypic profiling by characterization of cells imaged by fluorescence cytology and high-throughput analysis of a broad spectrum of biological attributes. Rapidly established as an efficient methodology for compound screening, HCS is a key technology in drug discovery because it prompts the investigation of changes in cell localization, intensity, texture or shape and hence allows the elucidation of discreet and physiologically applicable cellular processes such as cell or protein movement, morphological changes or protein modification [9, 11, second set of references]. In addition, mass spectrometry (MS) and nuclear magnetic resonance (NMR) spectroscopy are powerful analytical tools commonly used in the study of the chemical compositions of samples [12±15]. These techniques are often coupled to identify and quantify bioactive molecules in natural products with potential medicinal value [16±20]. High-performance liquid chromatography (HPLC) coupled with NMR spectroscopy has been used in a wide range of natural product studies, particularly to identify the chemical compositions of plant extracts [21, 22, second set of references].

### BRIEF DESCRIPTION OF THE INVENTION

The present invention recognizes that certain herbs, which have a long history of use by humans, may be effective in treating, inhibiting, preventing, reducing the incidence of, ameliorating or resolving a cancerous disease state or condition. To address this potential, three known Saudi Arabian herbs were examined for effectiveness as topoisomerase inhibitors.

The present invention recognizes further that the discovery process for biologically active small molecules, and their subsequent development into therapeutic compounds, can be optimized going forward by the application of automated microscopy, used in conjunction with image analysis to facilitate phenotypic profiling, based on the characterization of cells imaged by fluorescence cytology by a method called high-content screening (HCS). Rapidly established as a key methodology for next-stage compound screening, HCS is a high-throughput platform that uses image processing software to review images produced by automated fluorescence microscopy (Ghosh et al., 2004; Giuliano et al., 1997; Haney et al., 2006; Korn and Krausz, 2007; Nichols, 2006; Taylor, 2006). Quickly integrated as a key technology for drug discovery, the technique facilitates the high-throughput analysis of a broad spectrum of biological attributes, thereby permitting the investigation of changes in cell localization, intensity, texture or shape and hence allowing the elucidation of more discreet and physiologically applicable cellular processes such as cell or protein movement, morphological changes or protein modification (Nichols, 2006; Rausch, 2006).

Mass spectrometry (MS) and nuclear magnetic resonance (NMR) spectroscopy are common, powerful analytical tools for studying the chemical composition of samples (Diercks et al., 2001; Ge et al., 2010; Zhang et al., 2007; Zon and Robbins, 1983). These techniques have been coupled to identify and quantify bioactive molecules in natural products, such as bio-fluids and plant extracts, with potential medicinal value (Al-Talla et al., 2011; Emwas et al., 2015a; Emwas et al., 2015b; Keifer, 2000; Khorshid et al., 2015). Nowadays, high-performance liquid chromatography (HPLC) coupled with NMR spectroscopy has favorably been applied to a wide range of natural product studies, particularly to identify chemical compositions of plant extracts (Urban, 2006; Urban and Separovic, 2005).

In this study, the inventors used automated HCS to investigate bioactivity from fractions obtained from a selection of plants of the Saudi Arabian peninsula. They collected a range of plants that have traditionally been used as remedies for treating cancer and infectious diseases in Saudi Arabia. They tested the plants Anastatica hierochuntica (Kaff Maryam), Juniperus phoenicea (Arar), and Citrullus colocynthis (Hanzal), of which the two latter have previously been reported to have anticancer activities (Cairnes et al., 1980; Hussain et al., 2014; Tannin-Spitz et al., 2007). Using developed cytological profiles of small molecules with active compounds (known biological effects) and clustering them with plant fractions (unknown biological effects), the inventors selected the fractions expected to perturb human cancer cells and those clustered with known drugs that showed similar mechanisms. Plant fractions were then tested to determine their topoisomerase inhibition activity. Furthermore, chemical analyses of active fractions of J. phoenicea were preformed using liquid chromatography-MS (LC-MS), gas chromatography-MS (GC-MS), and NMR to gain insight into the active chemical compounds present in the plant.

The present invention therefore relates to extracts obtained from the group of herbs consisting of Anastatica hierochuntica (Kaff Maryam), Juniperus phoenicea (Arar), and Citrullus colocynthis (Hanzal) and mixtures thereof. Such herbs or chemical constituents thereof are anticancer agents, with topoisomerase inhibitor (Azar, Hanzal) and other anticancer activity (Kaff Maryam) and accordingly are effective in pharmaceutical compositions or nutritive supplements to treat, inhibit, prevent, reduce the incidence of, ameliorate and/or resolve cancerous disease states or conditions.

One or more extracts (aqueous, C₁-C₃ alcohol, including ethanol or aqueous C₁-C₃ alcohol, preferably water or aqueous ethanol extracts), pursuant to the present invention can be used alone or in combination with a pharmaceutically acceptable carrier, additive or excipient to treat, inhibit, prevent, reduce the incidence of, ameliorate and/or resolve a number of disease states or conditions including, for example, cancer. The herbal compositions of the present invention can be use to treat patients who have cancer or who are at risk for cancer, especially including recurrent and metastatic cancers.

The present invention also contemplates a method of treating, inhibiting, preventing, reducing the incidence of, ameliorating or resolving a cancerous disease state or condition, comprising administering to said patient an effective amount of a composition including an herb taken from the groups consisting of Anastatica hierochuntica (Kaff Maryam), Juniperus phoenicea (Arar), and Citrullus colocynthis (Hanzal) and mixtures thereof or an extract (aqueous, C₁-C₃ alcohol, including ethanol or C₁-C₃ aqueous alcohol, preferably water or aqueous ethanol extracts) obtained from one or more of these herbs.

The present invention contemplates a method for testing the potential effectiveness of medicinal herbal compositions in treating, inhibiting, preventing, reducing the incidence of, ameliorating or resolving a cancerous disease state or condition, the method comprising using automated microscopy in conjunction with image analysis to perform phenotypic profiling, including a characterizing of cells imaged by fluorescence cytology via high-content screening and comparing the results obtained against a standard (which is a predetermined value or values obtained from one or more compounds or compositions which exhibit activity against the cancer disease state or condition tested in order to provide a comparison of the activity of the herbal composition against the compound(s) or composition(s) of known activity). More particularly, this method comprises selecting one or more herbs suspected of possible anticancer activity; generating fractions of said one or more herbs; using high-content screening, measuring cytological profiles of said one or more herbs; comparing said cytological profiles with a set of reference compounds/compositions with known modes of action (a reference standard); and executing cluster analyses of the cytological profiles to determine modes of action of compounds in the generated fractions as topoisomerase inhibitors.

### DESCRIPTION OF THE DRAWINGS

FIGS. 1A and 1B illustrate analysis of core cellular features of HeLa cells in response to plant fractions. FIG. 1A is a set of three graphs heat map data for A. hierochuntica (ANA), C. colocynthis (CIT), and J. phoenicea (JUN) against the core cellular features. Individual plant fractions are presented on the y-axis and individual core cellular features are presented on the x-axis. Positive deviations from HeLa cells treated with plant fractions are displayed in yellow and negative deviations are displayed in blue. FIG. 1B is a set of three graphs summarizing the data for each plant with respect to the core cellular features.

FIG. 2 is a graph showing a cytological profiling heat map. Automated HCS was used to assess compound-related perturbations of human cells using a full set of cellular markers. To assign possible biological targets to the test compounds, the resulting cytological profiles were compared to profiles retrieved from reference compounds with known modes of action. Some of the plant fraction extracts closely matched FDA-approved anticancer drugs, clustering with topoisomerase inhibitors. Individual features are presented on the x-axis and individual compounds are presented on the y-axis.

FIGS. 3A-3C are graphs showing the cytotoxic effect of plant fractions on HeLa cells. FIGS. 3A and 3B show cells treated with various concentrations of plant extracts for 24 h or 48 h, stained with Hoechst, and assessed using HCS. Cells showed effects of cytotoxicity, which is indicator of induced apoptosis or necrosis. FIG. 3C shows the distribution of cells during the cell cycle: G2/M, S, G0/G1 and low phases after a 24 h treatment with plant fractions. Data shown are the means±SD.

FIGS. 4A and 4B are graphs showing the effect of plant fractions on mitochondrial superoxide production and cell membrane permeability. FIG. 4A shows HeLa cells treated with plant fractions for 24 h. An increase in the MitoSox signal was detected and correlated with the increasing plant fraction concentration. FIG. 4B shows HeLa cells treated with different concentrations of plant fractions for 24 h and then subjected to a cell membrane permeability test. Fluorescence readouts were normalized against an in-plate control. Each sample was tested in quadruplicate, and the data were presented as means±SD.

FIGS. 5A-5D are four graphs showing plant fractions induced an apoptotic effect on HeLa cells. HeLa cells were treated with several concentrations (1.56, 3.12, 6.25, 12.5, 25, and 50 μg/ml) of plant fractions for 24 h or 48 h. In the data depicted in FIGS. 5A and 5B automated HCS was used to measure the activity of caspase-9. In the data depicted in FIGS. 5C and 5D automated HCS was used to measure the activity of P53. The fluorescence readout was normalized against an in-plate control. Each sample was tested in quadruplicate, and the data are presented as means±SD.

FIG. 6 is a graph showing assessment of the double-strand breaks in the DNA. HeLa cells were treated with different concentrations of SPE fractions to detect the expression of γ-H2AX. The data are shown as means±SD.

FIG. 7 is a LC-MS chromatogram of a studied sample overlaid over an acetonitrile blank. Eight beaks were identify from LC-MS chromatogram (1) 185.98322 m/z (2) 144.98225 m/z (3) 144.98225 m/z (4) 288.28995 m/z (5) 256.26364 m/z (6) 387.18086 m/z (7) 415.21219 m/z (8) 637.30585 m/z

FIG. 8 is a pair of shared-abscissa graphs showing deconvolution of ion at m/z 185.98322, +ve ESI of analyte 1. Acesulfame-K, Chemical Formula: C₄H₅NO₄S

FIG. 9 is a pair of shared-abscissa graphs showing deconvolution of the ion at m/z 144.98158, +ve ESI of analyte 2 & 3. Ethephon, Chemical Formula: C₂H₆ClO₃P.

FIG. 10 is a pair of shared-abscissa graphs showing deconvolution of the ion at m/z 288.29000, +ve ESI of analyte 4. Sphinganine, Chemical Formula: C₁₇H₃₇NO₂

FIG. 11 is a pair of shared-abscissa graphs showing deconvolution of the ion at m/z 256.26355, +ve ESI of analyte 5. Palmitic amide, Chemical Formula: C₁₆H₃₃NO

FIG. 12 is a pair of shared-abscissa graphs showing deconvolution of the ion at m/z 387.18086, +ve ESI of analyte 6. Burseran or (+)Eudesmin, Chemical Formula: C₂₂H₂₆O₆

FIG. 13 is a pair of shared-abscissa graphs showing deconvolution of the ion at m/z 415.21219, +ve ESI of analyte 7. Estra-1,3,5(10)-triene-3,6beta,17beta-triol triacetate. Chemical Formula: C₂₄H₃₀O₆

FIG. 14 is a pair of shared-abscissa graphs showing deconvolution of the ion at m/z 637.30585, +ve ESI of analyte 8. Methyl 6-O-[2,3,4-tris-O-(2,2-dimethylpropanoyl)-6-methyl-β-D-glucopyranuronosyl]-β-D-galactopyranoside. Chemical Formula: C₂₉H₄₈O₁₅

FIG. 15 is a graph showing extended CH2 and CH3 region of NMR spectrum. The spectrum was recorded at room temperature using 600 MHz NMR spectrometer.

FIG. 16 is a graph showing extended OH and sugar region of NMR spectrum. The spectrum was recorded at room temperature using 600 MHz NMR spectrometer. This figure provides evidence of MS finding of the (Methyl 6-O-[2,3,4-tris-O-(2,2-dimethylpropanoyl)-6-methyl-β-D-glucopyranuronosyl]-β-D-galactopyranoside) molecules where the signals of several CH3 groups were observed around 1 ppm FIG. 2.

FIG. 17A shows the molecular structure of Estra-1,3,5(10)-triene-3,6beta,17beta-triol triacetate. FIG. 17B shows the molecular structure of Burseran. FIG. 17C shows the molecular structure of (+)Eudesmin. FIG. 17D is a graph providing evidence of MS finding of the extended aromatic region of NMR spectrum.

FIG. 18 shows a fragmentation pattern MS/MSn (n=2) of the identified compound (C₁₇H₃₈NO₂) at m/z 288.28970.

FIG. 19 shows a fragmentation pattern MS/MSn (n=2) of the identified compound (C₂₄H₃₁O₆) at m/z 415.21151.

FIG. 20 shows a fragmentation pattern MS/MSn (n=5) of the identified compound (C₂₈H₄₈O₁₅) at m/z 637.30659.

### DETAILED DESCRIPTION OF THE INVENTION

The following terms shall be used throughout the specification to describe the present invention. Where a term is not specifically defined herein, that term shall be understood to be used in a manner consistent with its use by those of ordinary skill in the art.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges that may independently be included in the smaller ranges are also encompassed within the invention, subject to any specifically excluded limit in the stated range. Where the stated range includes one or both of the limits, ranges excluding either both of those included limits are also included in the invention. In instances where a substituent is a possibility in one or more Markush groups, it is understood that only those substituents which form stable bonds are to be used. Where a substituent is not disclosed it is presumed (unless contrary to the underlying chemistry) that the substituent is a hydrogen atom.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

Furthermore, the following terms shall have the definitions set out below.

The term “patient” or “subject” is used throughout the specification within context to describe an animal, generally a mammal, especially including a domesticated animal (e.g. dog, cat, horse, pig, sheep, goat, etc.) and preferably a human, to whom treatment, including prophylactic treatment (prophylaxis), with the compounds or compositions according to the present invention is provided. For treatment of those infections, conditions or disease states which are specific for a specific animal such as a human patient, the term patient refers to that specific animal. In most instances, the patient or subject of the present invention is a human patient of either or both genders.

The term “effective” is used to describe an amount of a component, extract, material or solvent which is used to produce an intended effect in amount consistent the effect desired and may vary with the effect desired or which occurs.

The term “extract” is used to describe extract (an aqueous extract, a C₁-C₃ alcohol extract, including ethanol or aqueous C₁-C₃ alcohol extract, preferably water or aqueous ethanol extract) of one or more of the following herbs selected from the group consisting of Anastatica hierochuntica (Kaff Maryam), Juniperus phoenicea (Arar), and Citrullus colocynthis (Hanzal) and mixtures thereof.

Extracts of the present invention are prepared by exposing one or more of the herbs which are described above to an effective amount of an aqueous or alcohol solvent (an aqueous, C₁-C₃ alcohol, including ethanol or aqueous C₁-C₃ alcohol, preferably water or aqueous ethanol), preferably heated above room temperature, preferably at least 50° C. (including boiling) for a period of time (often between 1 minute to 48 hours or longer, often 15 minutes to 24 hours, often 20 minutes to 10 hours, often 25-30 minutes to 5 hours) effective to extract medicinal components of the herbs into the solvent. Extracts of solvents may be prepared using standard methods readily available in the art and may include the preferred methods of preparation as otherwise described herein.

The term “aqueous” is used to describe a solvent which comprises water in any amount. Preferably, extracts are provided using water or water/alcohol, preferably water/ethanol (at least about 50% by volume water within this mixture) solvent, preferably solvent which is heated (preferably boiled).

The term “C₁-C₃ alcoholic” or “C₁-C₃ alcoholic solvent” is used to describe a solvent which contains a C₁-C₃ alcohol in amounts greater than 50% and often greater than 95% (approaching up to 100%) by volume, often in combination with water. The term “ethanolic” or “ethanolic solvent” is used to describe a solvent which comprises ethanol in amounts greater than 50% and often greater than 95% (approaching up to 100%) by volume, often in combination with water. “Methanolic solvent” may also be used. As noted, the terms “alcoholic” and/or “ethanolic” may overlap with the term “aqueous” as otherwise defined herein.

The term “solid extract” is used to describe an extract of one or more of the herbs as otherwise disclosed herein which has been dried, dehydrated, lyophilized or otherwise solidified to avoid containing appreciable quantities of solvent, including water.

The term “compound”, as used herein, unless otherwise indicated, refers to any specific chemical compound disclosed herein and includes tautomers, regioisomers, geometric isomers, stereoisomers and where applicable, optical isomers (enantiomers) thereof, as well as pharmaceutically acceptable salts and derivatives (including prodrug forms) thereof. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds. The term also refers, within context, to prodrug forms of compounds which have been modified to facilitate the administration and delivery of compounds to a site of activity. The use of a bond presented as ----- signifies that a single bond is present or absent, depending on the context of the chemistry described. The use of a bond presented as [symbol] signifies that a single bond or a double bond is intended depending on the context of the chemistry described. It is understood by those of ordinary skill that molecules which are described herein are stable compounds as generally described hereunder.

The term “cancer” shall is used to describe a proliferation of tumor cells having the unique trait of loss of normal controls, resulting in unregulated growth, lack of differentiation, local tissue invasion, and/or metastasis. As used herein, neoplasms include, without limitation, morphological irregularities in cells in tissue of a subject or host, as well as pathologic proliferation of cells in tissue of a subject, as compared with normal proliferation in the same type of tissue. Additionally, neoplasms include benign tumors and malignant tumors (e.g., colon tumors) that are either invasive or noninvasive. Malignant neoplasms are distinguished from benign neoplasms in that the former show a greater degree of dysplasia, or loss of differentiation and orientation of cells, and have the properties of invasion and metastasis. The term cancer also within context, includes drug resistant cancers, including multiple drug resistant cancers and recurrent cancers which are often chemotherapy resistant. Examples of neoplasms or neoplasias from which the target cell of the present invention may be derived include, without limitation, carcinomas (e.g., squamous-cell carcinomas, adenocarcinomas, hepatocellular carcinomas, and renal cell carcinomas), particularly those of the bladder, bone, bowel, breast, cervix, colon (colorectal), esophagus, head, kidney, liver, lung, nasopharyngeal, neck, thyroid, ovary, pancreas, prostate, and stomach; leukemias, such as acute myelogenous leukemia, acute lymphocytic leukemia, acute promyelocytic leukemia (APL), acute T-cell lymphoblastic leukemia, adult T-cell leukemia, basophilic leukemia, eosinophilic leukemia, granulocytic leukemia, hairy cell leukemia, leukopenic leukemia, lymphatic leukemia, lymphoblastic leukemia, lymphocytic leukemia, megakaryocytic leukemia, micromyeloblastic leukemia, monocytic leukemia, neutrophilic leukemia and stem cell leukemia; benign and malignant lymphomas, particularly Burkitt's lymphoma, Non-Hodgkin's lymphoma and B-cell lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer (e.g., small cell lung cancer, mixed small cell and non-small cell cancer, pleural mesothelioma, including metastatic pleural mesothelioma small cell lung cancer and non-small cell lung cancer), ovarian cancer, testicular cancer, thyroid cancer, astrocytoma, esophageal cancer, pancreatic cancer, stomach cancer, liver cancer, colon cancer, and melanoma; mixed types of neoplasias, particularly carcinosarcoma and Hodgkin's disease; and tumors of mixed origin, such as Wilms' tumor and teratocarcinomas, among others. It is noted that certain epithelial tumors including ovarian, breast, colon, head and neck, medulloblastoma and B-cell lymphoma, among others are shown to exhibit increased autophagy and are principal target cancers for compounds and therapies according to the present invention.

The term “additional anti-cancer agent” is used to describe an additional compound which may be coadministered with one or more compounds of the present invention in the treatment of cancer. Such agents include, for example, everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhbitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanoliniumab, edotecarin, tetrandrine, rubitecan, tesmilifenc, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gcmcitabine, doxorubicin, irinotecan, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H -pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C11, CHIR-258,); 3-[5-(methylsulfonylpiperadinemethyl)-indolylj-quinolone, vatalanib, AG-013736, AVE-0005, the acetate salt of [D-Ser(Bu t) 6, Azgly 10 ] (pyro-Glu-His-Trp-Ser-Tyr-D-Ser(But)-Leu-Arg-Pro-Azgly-NH₂ acetate [C₅₉H₈₄N₁₈Oi₄-(C₂H₄O₂)ₓ where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, gleevac, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine; altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox,gefitinib, bortezimib, paclitaxel, irinotecan, topotecan, doxorubicin, docetaxel, vinorelbine, bevacizumab (monoclonal antibody) and erbitux, cremophor-free paclitaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, sspegfilgrastim, erythropoietin, epoetin alfa and darbepoetin alfa, ipilumumab, vemurafenib among others. Other anticancer agents which may be used in combination include immunotherapies such as ipilimumab, vemurafenib, pembrolizumab, nivolumab with the compounds of the present invention.

The terms “treat”, “treating”, and “treatment”, are used synonymously to refer within context to any action providing a benefit to a patient or subject at risk for or afflicted with a disease (cancer), including improvement in the condition through lessening or suppression of at least one symptom, delay in progression of the disease, prevention or delay in the onset of the disease, etc.

Treatment, as used herein, depending on context, encompasses both prophylactic and therapeutic treatment, principally of cancer. Compounds according to the present invention can, for example, be administered prophylactically to a mammal in advance of the occurrence of disease to reduce the likelihood of that disease or to reduce the likelihood of the spread of disease or its recurrence. Prophylactic administration is effective to reduce or decrease the likelihood of the subsequent occurrence/recurrence of disease in the mammal, or decrease the severity of disease that subsequently occurs, especially including metastasis of cancer or reduce the likelihood of the spread of disease. Alternatively, compounds according to the present invention can, for example, be administered therapeutically to a mammal that is already afflicted by disease. In one embodiment of therapeutic administration, administration of the present compounds is effective to eliminate the disease and produce a remission or substantially eliminate the likelihood of metastasis of a cancer. Administration of the compounds according to the present invention is effective to decrease the severity of the disease or lengthen the lifespan of the mammal so afflicted, in the case of cancer.

The term “standard” is used to describe a predetermined value obtained for a biological activity or other characteristic of a compound or composition with known activity or a known characteristic such that the predetermined value (reference) can be used to compare the activity or characteristics of the known compound or compositioin with the compound or composition of unknown activity or characteristic. Standards may be used inter alia, to determine the potential effectiveness of medicinal herbal compositions in treating, inhibiting, preventing, reducing the incidence of, ameliorating or resolving a cancerous disease state or condition, the phenotypic profile of a compound or composition, the impact on cells including the impact on cells imaged by fluorescence cytology via high-content screening, the cytological profiles of one or more compounds and/or herbs, the cytological profiles of compounds or compositions and cluster analyses of the cytological profiles to determine modes of action of compounds in the generated fractions including their action as topoisomerase inhibitors. Standards may also include the impact of a set of reference compounds with known modes of action for comparison purposes with compounds or compositions of unknown modes of action.

The term “pharmaceutically acceptable” as used herein means that the compound or composition is suitable for administration to a subject to achieve the treatments described herein, without unduly deleterious side effects in light of the severity of the disease and necessity of the treatment.

The term “inhibit” as used herein refers to the partial or complete elimination of a potential effect, while inhibitors are compounds that have the ability to inhibit.

The term “prevention” when used in context shall mean “reducing the likelihood” or preventing a disease, condition or disease state from occurring as a consequence of administration or concurrent administration of one or more compounds or compositions according to the present invention, alone or in combination with another agent. It is noted that prophylaxis will rarely be 100% effective; consequently the terms prevention and reducing the likelihood are used to denote the fact that within a given population of patients or subjects, administration with compounds according to the present invention will reduce the likelihood or inhibit a particular condition or disease state (in particular, the worsening of a disease state such as the growth or metastasis of cancer) or other accepted indicators of disease progression from occurring.

The term “topoisomerase” is used to describe topoisomerase I and II, which are enzymes which control changes in DNA by assisting in breaking and rejoining the backbone of DNA molecules through their phosphodiester linkages. The action of toposiomerases occurs during the normal cell cell. Inhibitors of these enzymes are thought to block the ligation process during the cell cycle, thus minimizing single and double stranded breaks in the DNA, resulting in maintaining the integrity of the genome and reducing the effects of such damage, which damage is believed to lead to cancer.

The present invention relates to herb extract compositions, especially solid extracts or extracts which are based at least in part on aqueous and/or C₁-C₃ alcoholic, preferably methanolic and/or ethanolic solvents of herbs selected from the group consisting of Anastatica hierochuntica (Kaff Maryam), Juniperus phoenicea (Arar), and Citrullus colocynthis (Hanzal) and mixtures thereof. Further aspects of the invention relate to compositions which comprise an effective amount of an herb extract in liquid, semi-solid or solid form, optionally in combination with a pharmaceutically acceptable carrier, additive or excipient. These compositions may be used to prevent, treat, ameliorate, or reduce the incidence of various cancerous disease states or conditions, comprising administering an effective amount of an extract as otherwise described herein to a patient in need thereof.

Pharmaceutical compositions according to the present invention comprise an effective amount of one or more compounds according to the present invention optionally in combination with a pharmaceutically acceptable additive, carrier or excipient.

In another aspect, the present invention is directed to the use of one or more extracts according to the present invention in a pharmaceutically acceptable carrier, additive or excipient at a suitable dose in an effective amount often ranging from about 0.05 to about 500 mg/kg of body weight per day or more, often about 0.1 to about 100 mg/kg/day, often within the range of about 0.1 to 50 mg/kg/day, most preferably in the range of 1 to 20 mg/kg/day. The desired dose may conveniently be presented in a single dose or as divided doses administered at appropriate intervals, for example as two, three, four or more sub-doses per day. It is noted that the extract may be diluted with water or pharmaceutically acceptable aqueous solvents for administration.

Ideally, the active ingredient should be administered to achieve effective peak plasma concentrations of the active compound preferably within the range of from about 0.05 to about 5 uM or higher. This may be achieved, for example, by oral or other route of administration as otherwise described herein. Oral dosages, where applicable, will depend on the bioavailability of the compounds from the GI tract, as well as the pharmacokinetics of the compounds to be administered. While it is possible that, for use in therapy, a compound of the invention may be administered as the raw chemical, it is preferable to present the active ingredient as a pharmaceutical formulation, presented in combination with a pharmaceutically acceptable carrier, excipient or additive.

Pharmaceutical formulations include those suitable for oral, rectal, nasal, topical (including buccal and sub-lingual), vaginal or parenteral (including intramuscular, sub-cutaneous and intravenous) administration. Compositions according to the present invention may also be presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives. When desired, the above described formulations may be adapted to provide sustained release characteristics of the active ingredient(s) in the composition using standard methods well-known in the art.

In the pharmaceutical aspect according to the present invention, the compound(s) according to the present invention is formulated preferably in admixture with a pharmaceutically acceptable carrier. In general, it is preferable to administer the pharmaceutical composition orally, but certain formulations may be preferably administered parenterally and in particular, in intravenous or intramuscular dosage form, as well as via other parenteral routes, such as transdermal, buccal, subcutaneous, suppository or other route, including via inhalation intranasally. Oral dosage forms are preferably administered in tablet or capsule (preferably, hard or soft gelatin) form. Intravenous and intramuscular formulations are preferably administered in sterile saline. Of course, one of ordinary skill in the art may modify the formulations within the teachings of the specification to provide numerous formulations for a particular route of administration without rendering the compositions of the present invention unstable or compromising their therapeutic activity.

In particular, the modification of the present compounds to render them more soluble in water or other vehicle, for example, may be easily accomplished by minor modifications (such as salt formulation, etc.) which are well within the ordinary skill in the art. It is also well within the routineer's skill to modify the route of administration and dosage regimen of a particular compound in order to manage the pharmacokinetics of the present compounds for maximum beneficial effect to the patient.

Formulations containing the compounds of the invention may take the form of solid, semi-solid, lyophilized powder, or liquid dosage forms, such as, for example, tablets, capsules, powders, sustained-release formulations, solutions, suspensions, emulsions, suppositories, creams, ointments, lotions, aerosols or the like, preferably in unit dosage forms suitable for simple administration of precise dosages.

The compositions typically include a conventional pharmaceutical carrier, additive or excipient and may additionally include other medicinal agents, carriers, and the like. Preferably, the composition will be 0.001% to 95+% by weight of an extract or extracts of the invention, preferably 0.05% to 75-80% by weight of an extract or extracts of the invention, with the remainder consisting of suitable pharmaceutical additives, carriers and/or excipients. For oral administration, such excipients include pharmaceutical grades of mannitol, lactose, starch, magnesium stearate, sodium saccharine, talcum, cellulose, glucose, gelatin, sucrose, magnesium carbonate, and the like. If desired, the composition may also contain minor amounts of non-toxic auxiliary substances such as wetting agents, emulsifying agents, or buffers.

Additional pharmaceutically acceptable carriers that may be used in the pharmaceutical compositions of this invention include, but are not limited to, ion exchangers, alumina, aluminum stearate, lecithin, self-emulsifying drug delivery systems (SEDDS) such as a-tocopherol, polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropyleneblock polymers, polyethylene glycol and wool fat. Cyclodextrins such as α-, β-, λ-cyclodextrin or chemically modified derivatives such as hydroxyalkylcyclodextrins, including 2- and 3-hydroxypropyl-cyclodextrins, or other solubilized derivatives may also be advantageously used to enhance delivery of the compounds of the invention.

Liquid compositions can be prepared by dissolving or dispersing the extracts in liquid, semi-solid or solid form (about 0.5% to about 20%), and optional pharmaceutical additives, in a carrier, such as, for example, aqueous saline, aqueous dextrose, glycerol, or ethanol, to form a solution or suspension. For use in oral liquid preparation, the composition may be prepared as a solution, suspension, emulsion, or syrup, being supplied either in liquid form or a dried form suitable for hydration in water or normal saline.

When the composition is employed in the form of solid preparations for oral administration, the preparations may be tablets, granules, powders, capsules or the like. In a tablet formulation, the composition is typically formulated with additives, e.g. an excipient such as a saccharide or cellulose preparation, a binder such as starch paste or methyl cellulose, a filler, a disintegrator, and other additives typically used in the manufacture of medical preparations.

An injectable composition for parenteral administration will typically contain the compound in a suitable i.v. solution, such as sterile physiological salt solution. The composition may also be formulated as a suspension in a lipid or phospholipid, in a liposomal suspension, or in an aqueous emulsion.

The pharmaceutical compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Methods for preparing such dosage forms are known or will be apparent to those skilled in the art; for example, see “Remington's Pharmaceutical Sciences” (17th Ed., Mack Pub. Co, 1985). The person of ordinary skill will take advantage of favorable pharmacokinetic parameters of the pro-drug forms of the present invention, where applicable, in delivering the present compounds to a patient suffering from a viral infection to maximize the intended effect of the compound.

The pharmaceutical compositions according to the invention may also contain other active ingredients in the treatment of any one or more of the disease states or conditions which are treated with herbal extracts according to the present invention. Effective amounts or concentrations of each of the active compounds are to be included within the pharmaceutical compositions according to the present invention.

The individual components of such combinations may be administered either sequentially or simultaneously in separate or combined pharmaceutical formulations.

The term “coadministration” is used to describe the administration of two or more active compounds, in this case a compound according to the present invention, in combination with an additional anti-cancer, antiviral agent (especially where the virus is implicated in cancer, such as Heptatitis B virus “HBV” or Hepatitis C virus “HCV”) or other biologically active agent, in effective amounts. Although the term coadministration preferably includes the administration of two or more active compounds to the patient at the same time, it is not necessary that the compounds actually be administered at the exact same time, only that amounts of compound will be administered to a patient or subject such that effective concentrations are found in the blood, serum or plasma, or in the relevant tissue at the same time.

When one or more of the compounds according to the present invention is used in combination with a second therapeutic agent active the dose of each compound may be either the same as or differ from that when the compound is used alone. Appropriate doses will be readily appreciated by those skilled in the art.

In method aspects according to the present invention, one or more pharmaceutical compositions according to the present invention may be administered to a patient in the treatment or prevention of any disease state or condition previously mentioned. An effective amount of an herbal extract as otherwise described herein is administered to a patient exhibiting symptoms of a disease state or condition as otherwise described herein in order to treat the symptoms of the disease states and/or conditions and reduce or eliminate the likelihood that the disease state or condition will deteriorate.

Pharmaceutical compositions according to the present invention comprise an effective amount of one or more of the extracts in liquid, semi-liquid or solid form, otherwise described herein, optionally in combination with a pharmaceutically acceptable carrier, additive or excipient, and further optionally in combination with at least one additional agent useful in treating a disease state or condition which is related to or modulated through NRF2 protein. In this aspect of the invention, multiple compounds may be advantageously formulated to be coadministered for the prophylactic and/or thereapeutic treatment of any one or more of the disease states or conditions described hereinabove.

The individual components of such combinations as described above may be administered either sequentially or simultaneously in separate or combined pharmaceutical formulations. When one or more of the extracts according to the present invention is used in combination with a second therapeutic agent active the dose of each may be either the same as or differ from that when the compound is used alone. Appropriate doses will be readily appreciated by those skilled in the art.

### Materials and Methods

TABLE 1
Summary information about subject plants

| Scientific | | Traditional | |
|---|---|---|---|
| name | Family name | name | Code |
| Citrullus | Cucurbitaceae | Hanzal | CIT |
| colocynthis | | | |
| (L.) Schrad. | | | |
| Anastatica | Brassicaceae | Kaff | ANA |
| hierochuntia | | Maryam | |
| L. | | | |
| Juniperus | Cupressaceae | Arar | JUN |
| phoenicea | | | |
| Pall. | | | |

### Plant Collection, Identification, and Extraction

Dried Citrullus colocynthis (Source origin: seeds, description of appearance: dark brown, about 8 mm long and 2 mm thick), Juniperus phoenicea, and Anastatica hierochuntica were purchased from herbalists in the Jeddah region of Saudi Arabia. The taxonomic identity of each plant was confirmed by a Saudi taxonomist (see Table 1, above). The plants were washed, crushed in liquid nitrogen, and macerated at 10% in dichloromethane:methanol (1:1) overnight at room temperature. Subsequently, samples were centrifuged at 13,000 g for 30 min to remove particulate material. Extracts were fractionated using solid-phase extraction (SPE) with Bond Elut C2, PPL, C18, or CN-E columns (Agilent Technologies). Columns were conditioned before fractionation with 1 ml of methanol, 5 ml of CHROMASOLVE water, and 1 ml of acidified CHROMASOLVE water (pH 2) and then 3 ml of the extract was loaded into the column and eluted with 500 μl of a gradually non-polar 10-100% water:methanol gradient (10% step size). The properties of the extracted materials were used to help pre-select specific chemical structures (aliphatic/aromatic) and chemical properties (polar/non-polar) of the potentially active compounds (Table 2). Fractions were dried under a vacuum (CentriVap Complete, Labconco, Kansas City, Mont., USA).

TABLE 2
Full description of Solid phase extraction cartridges (SPE-Cartridges) for extraction
of plants natural products. This figure were adapted from the cited publication[1]

| Bond Elut | | | Primary | |
|---|---|---|---|---|
| SPE- | | | Retention | Typical Sample |
| Cartridges | Type of Material | Properties | Mechanism | Types |
| C₂ | Silica based, ethyl | Alternative | Weakly | Plasma, urine, |
| | bonded, endcapped | sorbent, if | nonpolar | aqueous samples |
| | | analytes are | | |
| | | retained too | | |
| | | strongly on C₈ | | |
| | | or C₁₈ phases | | |
| C₁₈ | Silica based, | Extreme | Strongly | Water, aqueous |
| | trifunctional octadecyl | retentive nature | nonpolar | biological fluids |
| | bonded, endcapped | for nonpolar | | |
| | | compounds, | | |
| | | applicable for | | |
| | | desalting | | |
| | | aqueous | | |
| | | matrices | | |
| CN-E | Silica based, | Different | Moderately | Aqueous samples |
| | cyanopropyl | selectivity to | nonpolar | (nonpolar), organic |
| | bonded, endcapped | alkyl and | (aqueous | samples (polar) |
| | | aliphatic | matrix) or | |
| | | functionalized | polar | |
| | | phases due to | (nonpolar | |
| | | electron density | organic | |
| | | of the aromatic | matrix) | |
| | | ring | | |
| PPL | Styrene- | Extreme | Highly polar | Waste water |
| | divinylbenzene (SDVB) | hydrophobicity | | (phenols) |
| | polymer with a | and surface | | |
| | proprietary derivitized | area, achieves | | |
| | nonpolar surface | high recovery | | |
| | | levels and fast | | |
| | | extraction | | |
| | | speeds | | |

### Cell Culture and Compound Transfer

HeLa cells (parental HeLa cell line, NIH AIDS reagents and reference program) were cultured under typical conditions at 37° C. under 5% CO₂. HeLa cells were plated in 384-well clear-bottomed black plates (Greiner bio-one Germany) at a density of 2,000 cells per well in 25 μl of Dulbecco's modified Eagle's medium (DMEM containing GlutaMAX-1; 4.5 g/L D-Glucose; Pyruvate; Gibco, Darmstadt, Germany). The cells were incubated for 24 h at 37° C. under 5% CO₂. Natural product SPE compounds were diluted in DMEM, and 100 μl of the diluted compound was transferred to 4 of the 384 wells (25 μl per well) to a 10-mM final concentration per well. The plates were then transferred to 37° C. for 24 h.

### High-Content Screening Imaging and Analysis

After 24 h of incubation, the plates (see above) were treated with various fluorescent stains and antibodies (R. Voolstra, 2016). For each SPE fraction, we aimed to assay 10 cellular organelles and regulatory proteins. To avoid overlap between fluorescent stains, four different panel stains were used for each sample. Plates were imaged using an ArrayScan™ VTI HCS reader (Cellomics, Thermo Fisher Scientific) with a 10× Zeiss objective lens. Images were analyzed with the Compartmental Analysis BioApplication (Cellomics, Thermo Fisher Scientific) for a minimum of 500 valid objects. Background correction was applied to all the images before being quantified. Panels were as follows: 1) ER, lysosome, and membrane; (2) nucleus, P53, and caspase-9; (3) nucleus, mitochondria, cytochrome C, and NF-κB; (4) nucleus, actin, and tubulin. Cells were stained by conducting permeabilization, blocking, and washing steps. The following HCS reagents were used: wash buffer I, wash buffer II, blocking buffer, and permeabilization buffer (Cellomics, HCS reagents, Thermo Fisher Scientific). Stain specific information and incubation times are set forth in Table 3. Subsequently, all plates were washed three times with wash buffer, sealed and stored at 4° C. until further use. Measurements from the reader were averaged, converted to feature scores, clustered, and analyzed using the multiple experiment viewers option by hierarchical clustering and Pearson's correlation (Saeed et al., 2003). For each of the tested fractions cytological profile was produced for each compound. In total, 21 core cellular features were selected from 12 cellular markers. The difference between the treated and control values for each feature was normalized to score between −1 and 1. Control wells were incubated with only pure DMEM (no fractions). HCS profiling and analysis followed (R. Voolstra, 2016).

TABLE 3
The panel's description for the cellular features measured in cytological profiling.

| | Cellular | | | Incubation |
|---|---|---|---|---|
| Panels | feature | Fluorescent stain | Secondary Antibody | Time |
| Panel 1 | Nucleus | Hoechst Stain | — | 10 min |
| | | (OG1726671-Thermo | | |
| | | Scientific) | | |
| | ER | ER tracker | | 30 min |
| | Lysosome | Lyso tracker | | 30 min |
| Panel 2 | Nucleus | Hoechst Stain | GAR550(OC183252)- | 10 min |
| | | (OG1726671-Thermo | GAR488(OC183252) | |
| | | Scientific) | | |
| | p53 | P53 (MA512557-Thermo | | 1 hour |
| | | Scientific) | | |
| | Caspase 9 | Cleaved Caspase-9 | | 1 hour |
| | | antibody | | |
| | | (ASP315- Thermo | | |
| | | Scientific) | | |
| Panel 3 | Nucleus | Hoechst Stain | GAR488(OI189170)- | 10 min |
| | | (OG1726671-Thermo | GAM650 | |
| | | Scientific) | | |
| | Mitochondria | MitoTracker Orange | | 30 min |
| | | CMTMRos (M7510, life | | |
| | | technologies) | | |
| | NFkB | NFkappaB/p65 Antibody | | 1 hour |
| | | (PA5-16545) | | |
| | Cytochrome C | Cytochrome C Antibody | | 1 hour |
| | | (MA5-11823-Thermo | | |
| | | Scientific) | | |
| Panel 4 | Nucleus | Hoechst Stain | GAM550 (NJ172004) | 10 min |
| | | (OG1726671-Thermo | | |
| | | Scientific) | | |
| | Actin | Phalloidin-FITC | | 1 hour |
| | Tubulin | Beta-3 Tubulin Antibody | | 1 hour |
| | | (MA1-19187) | | |

### Cell Loss and Cell Cycle Analysis

HeLa cells were seeded into 384 wells, treated with various concentrations (0, 1.56, 3.12, 6.25, 12.5, 25, 50 μg//ml of plant fractions (24 μl/well), and maintained under culturing conditions for 24 h or 48 h. Cells were fixed with 3.7% formaldehyde for 15 min, washed twice with Dulbecco's phosphate-buffered saline (DPBS), and stained with Hoechst 33342 (OG1726671-Thermo Scientific) prepared in DPBS (1 mg/ml) for 10 min in the dark at room temperature. Staining allowed us to quantify the DNA content and to determine cell numbers using the HCS reader (Cellomics, Thermo Fisher Scientific) and the BGRFR 386-23 filter set. Cell loss was calculated as the percentage of optical density (OD) of the treated cells compared to the negative control (untreated cells):

$$\% Cell Loss = \frac{OD value of experimental sample ( treated cells )}{OD value of experimental sample ( Negative control cells )} \times 10 \text{?}$$

$$\text{?} \text{indicates text missing or illegible when filed}$$

The Cell Cycle Bio Application (Cellomics, Thermo Fisher Scientific) automatically categorizes each cell's total nuclear intensity into one cell cycle phase. Cells categorized as having DNA˜2N, 2N<DNA<4N, or DNA˜4N were assigned the cell cycle phases G0/G1, S, or G2/M, respectively. Cells categorized as DNA<2N or DNA>4N were considered to be damaged/apoptotic (low nuclear intensity value) or clumped/higher ploidy, respectively (high nuclear intensity value). Cells were treated with 25 μg/ml of the appropriate SPE fraction.

### MitoSOX and Membrane Permeability Test

MitoSox Red (M36008, Life Technologies), an indicator of mitochondrial superoxide production, and cell membrane permeability dye (V35123-ThermoScientific) were prepared according to the manufacturer's instructions. Cells were incubated with 5-mM MitoSOX Red and 2-mM cell membrane permeability dye for exactly 20 min at 37° C. in 5% CO₂ in the dark. The resulting labeled cells were washed gently with phosphate buffer saline (PBS) to remove any excess unbounded dye. Cells were fixed with 4% formaldehyde for 20 min, washed twice with PBS, and stained with Hoechst33342 stain for 10 min. Cells were evaluated on the HCS reader using the following filter settings: BGRFR 485-20 for the permeability dye, BGRFR 549-15 for MitoSOX, and BGRFR 386-23 for Hoechst.

### Caspase-9 Activity and the P53 Assay

A time-dependent study of caspase-9 and of P53 activities were performed in triplicate using the HCS reader. Cells were treated with various concentrations of plant fractions (25 μl/well) for 24 h or 48 h under the culturing conditions described. Next, cells were fixed with 3.7% formaldehyde for 15 min and washed twice with DPBS. Fixed cells were permeabilized with 0.1% Triton X-100 in PBS for 17 min and washed twice with DPBS. Samples were blocked for 30 min and incubated with cleaved caspase-9 (ASP315-Thermo Scientific) and P53 antibodies (MA512557-Thermo Scientific) for 1 h. Samples were washed three times with wash buffer II (prepared in water), washed twice with DPBS, and incubated with goat anti-rabbit IgG550 (GAR-DyLight 550-84541) and goat anti-mouse IgG 488 (GAM-DyLight 488-35502) secondary antibodies for 1 h. Cells were rinsed three times with wash buffer II, and nuclei were stained with Hoechst 33258 (OG1726671-Thermo Scientific). Next, cells were washed twice with DPBS and 25 μl of PBS. Stained cells were visualized and the images were captured using the HCS reader (Cellomics, Thermo Fisher Scientific). Primary and secondary antibodies were prepared in blocking buffer. A cell profiling bioapplication module was used to quantify the fluorescence intensities of each dye: BGRFR 485-20 for P53, BGRFR 549-15 for Caspase-9, and BGRFR 386-23 for Hoechst.

### Histone H2AX Phosphorylation

Cell culturing and preparing were performed as described earlier. Cells were treated with various concentrations of plant fractions (25 μl/well) for 6 h and cultured under the conditions described earlier. Next, cells were fixed with 3.7% formaldehyde for 15 min and washed twice with DPBS. Fixed cells were penneabilized with 0.1% Triton X-100 in PBS for 15 min and washed twice with DPBS. Samples were blocked with 2% fetal bovine serum for 15 min and incubated with anti-Histone H2AX polyclonal A (PA184856-Thermo Scientific) for 1 h. Samples were washed three times with wash buffer II (prepared in water) and washed twice with DPBS. Cells were incubated with goat anti-mouse488 (GAM-DyLight 488 84540) secondary antibodies for 1 h, rinsed three times with wash buffer II, and the nuclei were stained with Hoechst 33342. Finally, cells were washed twice with DPBS and 25 μl of PBS.

Stained cells were visualized and their images were captured using the HCS reader. Primary and secondary antibodies were prepared in blocking buffer. A cell profiling bioapplication module was used to quantify the fluorescence intensities of each dye: BGRFR 485-20 for H2AX and BGRFR 386-23 for Hoechst.

### HPLC LTQ Orbitrap Mass Spectrometry Analysis

To separate the extracted natural products, the inventors used a C18 (ZORBAX ECLIPS XDB, 5μ, 4.6×250 mm) column (Agilent Technologies) with a gradient composed of water/acetonitrile to achieve the most highly resolved chromatography. The mobile phase solvents were composed of A: 100% water+(0.1% formic acid) and B: 100% acetonitrile+(0.1% formic acid); the injection volume was 10 μL, and the flow rate was set to 450 μL/min. Xcalibur™ software (Thermo Scientific) was used to develop and treat the data. We used a Thermo LTQ Velos Orbitrap mass spectrometer (Thermo Scientific) equipped with an electrospray ionization source. The mass scan range was set to 100-2000 m/z with a resolving power of 100 000. The m/z calibration of the LTQ-Orbitrap analyzer was performed in the positive electrospray ionization mode using a solution containing caffeine, MRFA (met-arg-phe-ala) peptide, and Ultramark 1621 according to the manufacturer's guidelines. We performed this analysis with a heated ion source equipped with a metal needle and operated at 4 kV. The source vaporizer temperature was adjusted to 350° C., the capillary temperature was set at 250° C., and the sheath and auxiliary gases were optimized and set to 40 and 20 arbitrary units, respectively. The bioactive fraction of J. phoenicea was identified by considering the measured mass and the mass provided by online software, such as Metlin, MetFrag, and Chemspider. To confirm the identity of the product identified, MS/MS studies were performed.

### Chemical Analysis by Gas Chromatography-Mass Spectrometry

GC-MS was performed on 20% eluted methanol fractions of J. phoenicea on C2 column cartridges. The setup comprised an Agilent 7890A GC system with split injection (280 C; 10:1) coupled to an Agilent MS model 5975C with triple-axis detector (Agilent Technologies, USA) with a HP-5MS capillary column (30 m×250 m; film thickness: 0.25 m) (Agilent Technologies, USA). The gas chromatography began with an oven temperature of 50° C. for 1 min, which increased 300° C. for 35 min under a constant helium pressure (10 psi). Samples were dissolved in methanol and a 1 μl aliquot was injected automatically. Compounds were identified by matching their EI-MS spectra with those in NIST 2011 Mass Spectral Library using MSD ChemStation (Agilent Technologies)

### Nuclear Magnetic Resonance Analysis

The sample was prepared by dissolving the bioactive fractions in 600 μl of deuterated water, D₂O, and then 550 μl of the solution was transferred to 5-mm NMR tubes. NMR spectra were acquired using a Bruker 600 AVANAC III spectrometer equipped with a Bruker broad band observe multinuclear probe (BrukerBioSpin, Rheinstetten, Germany). To achieve a high signal to noise ratio, the ¹H NMR spectra were recorded by collecting 4 k scans with a recycle delay time of 2 s. To suppress the water peak, each spectrum was induced by an excitation sculpting pulse sequence using a standard (zgesgp) program from the Bruker pulse library. The free induction decay (FID) data were collected with a spectral width of 18028 Hz digitized into 32 k data points, and the FID signals were zero-filled and amplified by an exponential line-broadening factor of 1 Hz before Fourier transformation. Bruker Topspin 2.1 software was used in all experiments to collect and analyze the data.

### Statistical Analysis

All statistical analyses were performed using GraphPad Prism Version 6 (GraphPad Software, La Jolla, Calif.). All data are representative of at least four replicates, and data are means±SD unless otherwise indicated. Statistical significance of the comparison between two groups was determined by a two-tailed Student's t-test where indicated. Significant differences were considered at p-values of ≤0.05.

### Results

### Cytological Profiling of Natural Plant Products from Saudi Arabia

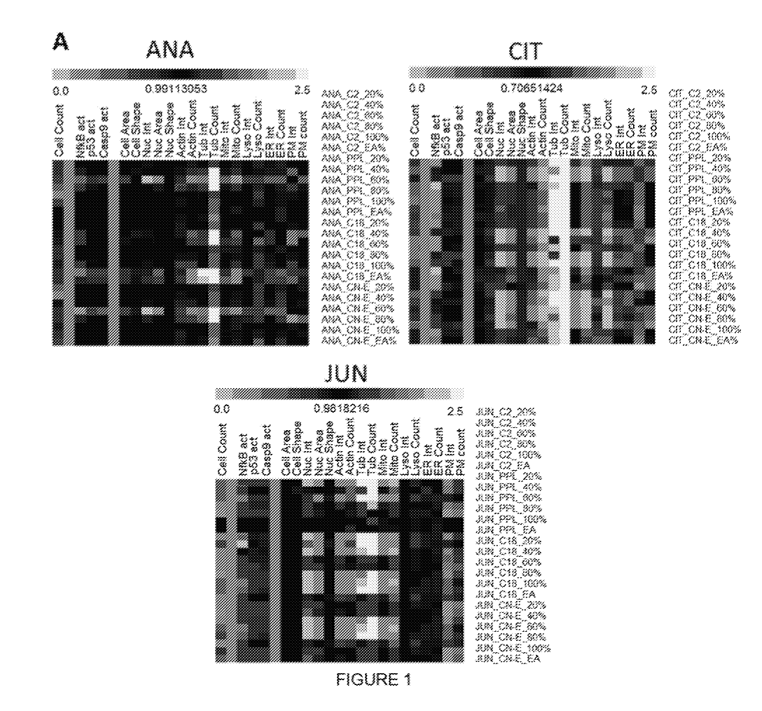

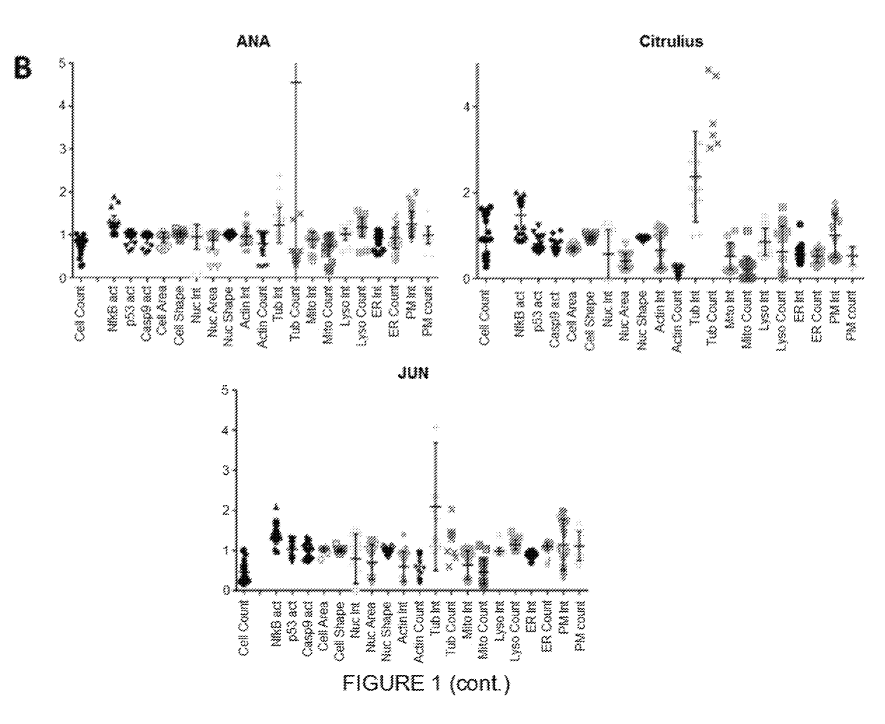

A total of 84 natural products fractions were obtained in triplicates, screened, and feature scores were calculated in relation to internal controls on each plate. The inventors used a set of core cellular features (R. Voolstra, 2016) to reduce the dimensional space defined by a set of factors reflecting the major underlying phenotypic attributes (see Table 4, below). All three plants showed a positive response to the marker tubulin (FIG. 1). C. colocynthis (CIT) and J. phoenicea (JUN) heat map profiles showed a similar effect on some markers: a negative effect on mitochondria, actin, nuclear area, and intensity and a positive effect on NF-κB. A. hierochuntica (ANA) was generally active with fractions high in methanol, but cell counts were lower at the higher solvent concentrations found in C2 and C18 fractions. CIT had no significant affect on cell number while JUN reduced cell number significantly in most fractions. ANA fractions eluted from C2 with 60% methanol, C18 with 60% and 80%, and CN-E with 40% methanol cartridges had a negative effect on nuclear intensity and area, mitochondria, actin, and endoplasmic reticulum. CIT fractions showed a very strong negative affect on lysosomes and slightly negative effect on endoplasmic reticulum and membrane permeability signal. All JUN active fractions showed minor negative effects on membrane permeability.

Table 4: Core Cell Feature Markers with Parameter Measurements and Phenotypic Attributes

[symbol]

### Cytological Profiling of Plant Fractions in Comparison to Reference Compounds

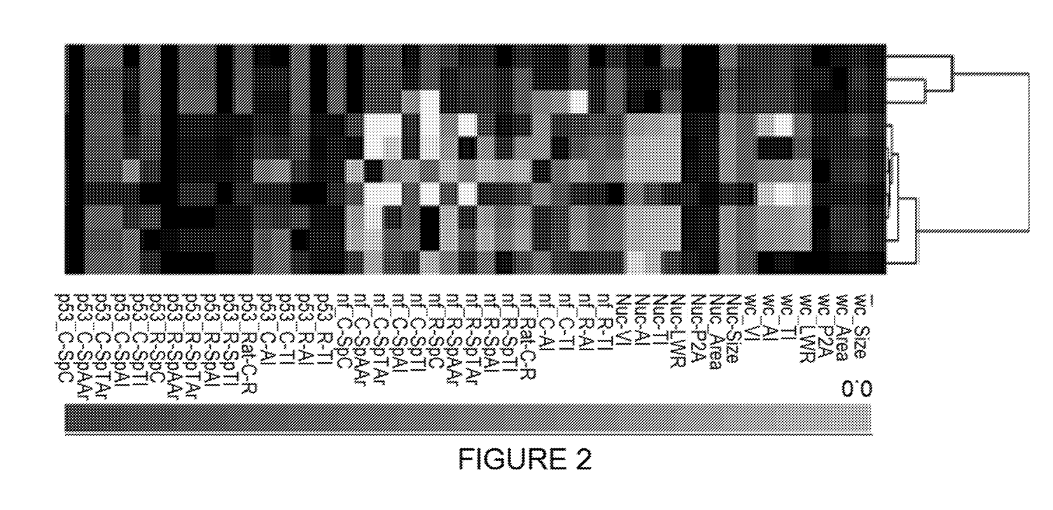

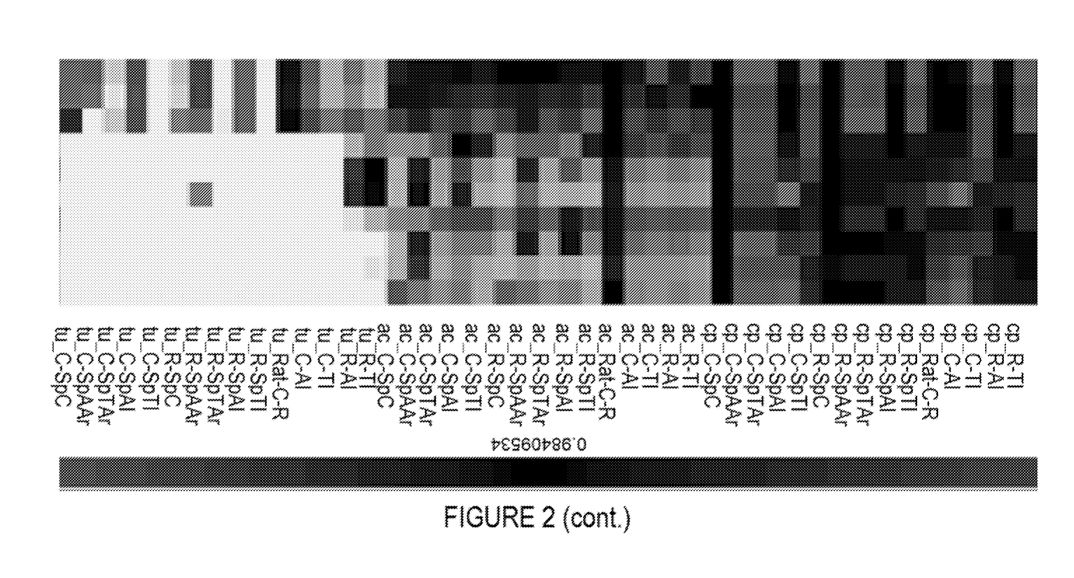

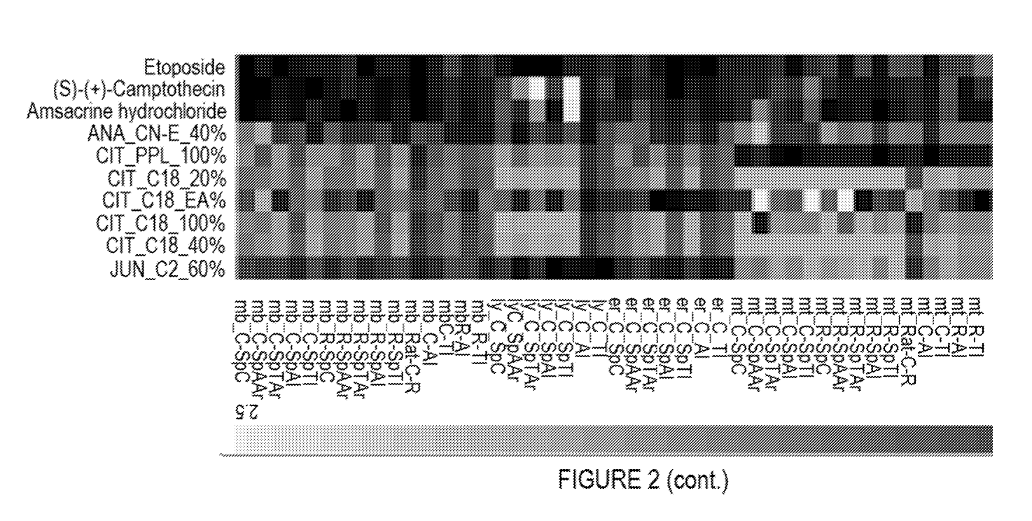

Possible biological targets were assigned to test unknown fractions by comparing similarities of high-resolution cytological profiles (consisting of more than 130 cellular features) of plant fractions to reference compounds with known modes of action. The library of reference compounds contains 735 compounds (LOPAC1280) that affect a variety of known cellular targets. including apoptosis, G proteins and cyclic nucleotides, gene regulation and expression, ion channels, lipid signaling, multi-drug resistance, neurotransmission, and phosphorylation. The reference compound library was screened, stained, and analyzed using the same method applied to the plant fractions, which previously discussed (R. Voolstra, 2016). Cluster analysis generated data both from reference compounds and plant fractions, where several fractions closely matched FDA-approved anticancer drugs, including the topoisomerase inhibitors etoposides, carnptothecin and amsacrine hydrochloride (FIG. 2).

### Assessment of Cell Loss and Cell Cycle Arrest

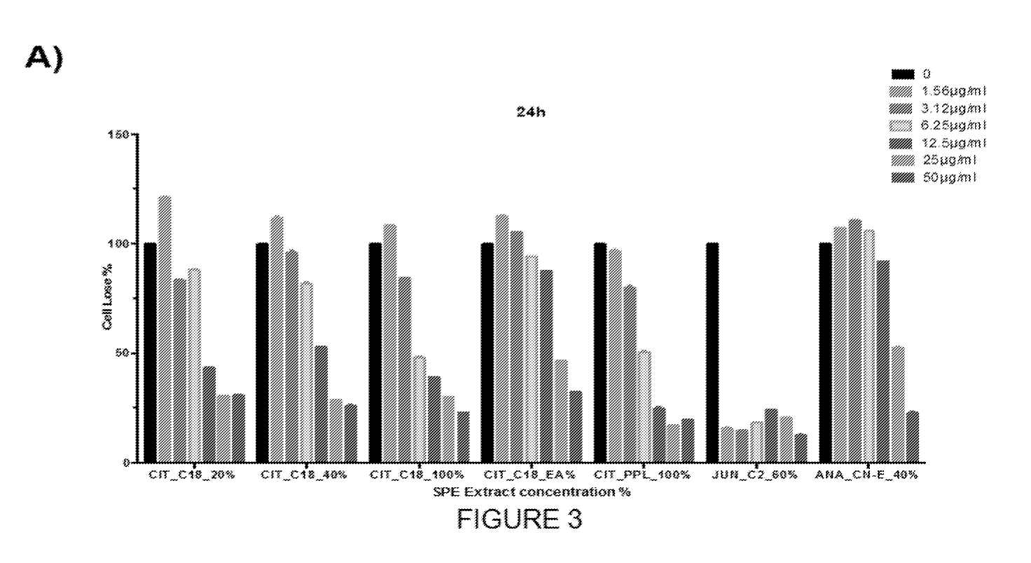

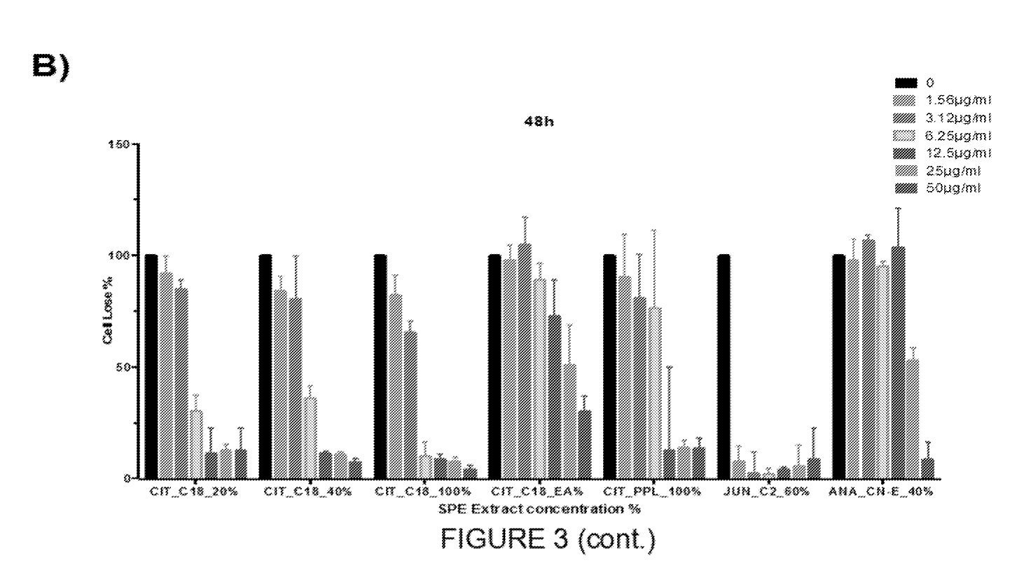

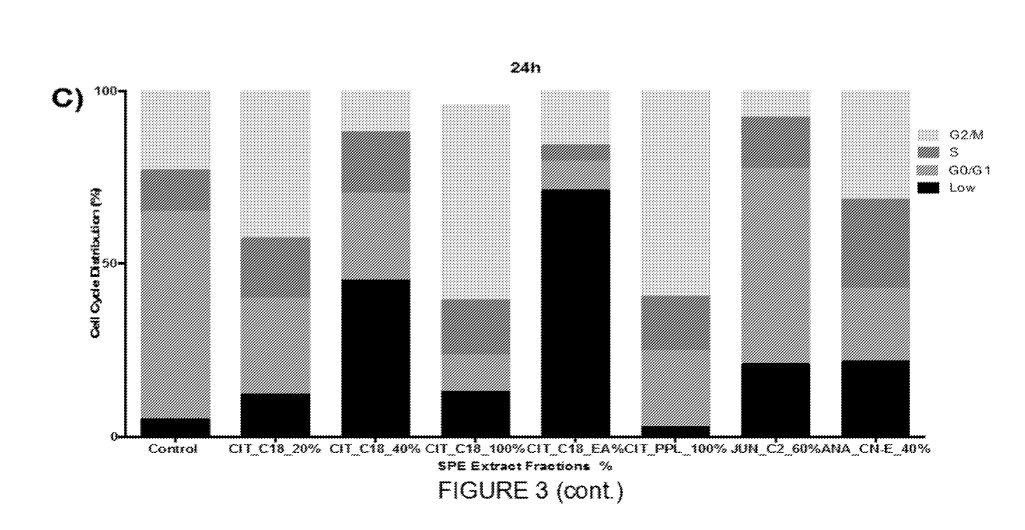

Cytotoxic anticancer drugs have the potential to elicit cancer cell death by apoptosis or cell necrosis. To evaluate the anticancer effect of SPE fractions, HeLa cells were incubated with various concentrations of plant cell fractions (1.56, 3.12, 6.25, 12.5, 25, and 50 μg/ml) for 24 h or 48 h. After 24 h of incubation, CIT plant fractions caused a loss of cells at 12.5 μg/ml unlike the fraction ANA_CN-E_40%, which only caused a loss of cells at higher concentrations (25 and 50 μg/ml). The most prominent decrease in cell number even at the lowest concentration (1.56 μg/ml) was evident from the JUN_C2_60% fraction (FIG. 3A). After 48 h of incubation, with the exception of CIT_C18_EA%, which reduced cell number only at 12.5 μg/ml, all CIT plant fractions caused cell loss at 6.25 μg/ml. ANA_CN-E_40% showed toxicity only at the highest concentrations (25 and 50 μg/ml), while JUN_C2-60% had the overall highest cytotoxicity effect on HeLa cells (FIG. 3B).

Next, the inventors investigated whether the plant fractions affected the cell cycle. The data generated suggest that several extracted fractions induced cell cycle arrest at different phases (FIG. 3C). Results indicated that plant fractions increased the percentage of Sub-G1 cells, an indicator of apoptotic cells. Generally, fractions of C. colocynthis and ANA_CN-E_40% strongly induced cell cycle arrest in the G0/G1 phase. CIT_C18_EA% strongly induced cell cycle arrest in the S phase and JUN_C2_60% strongly induced cell cycle arrest in the G2/M phase.

### MitoSOX and Membrane Permeability

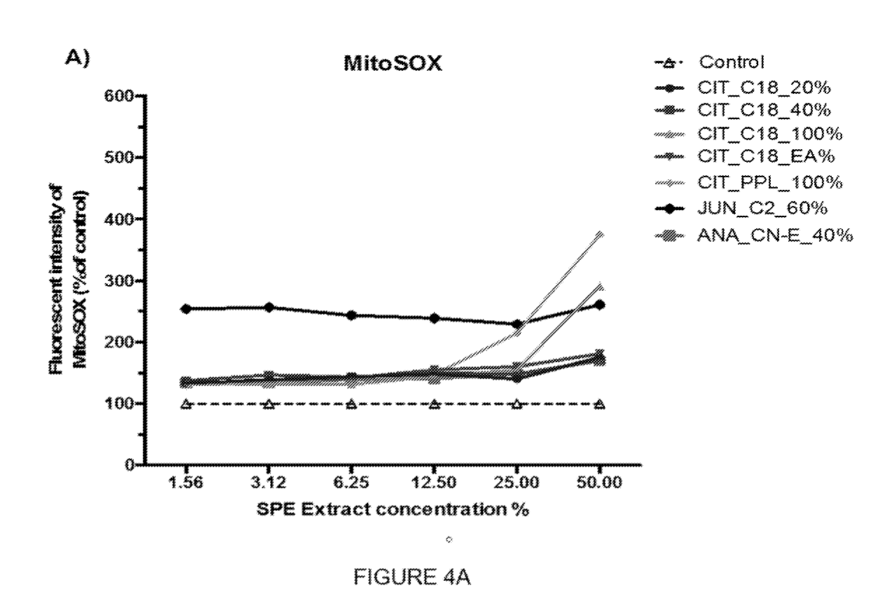

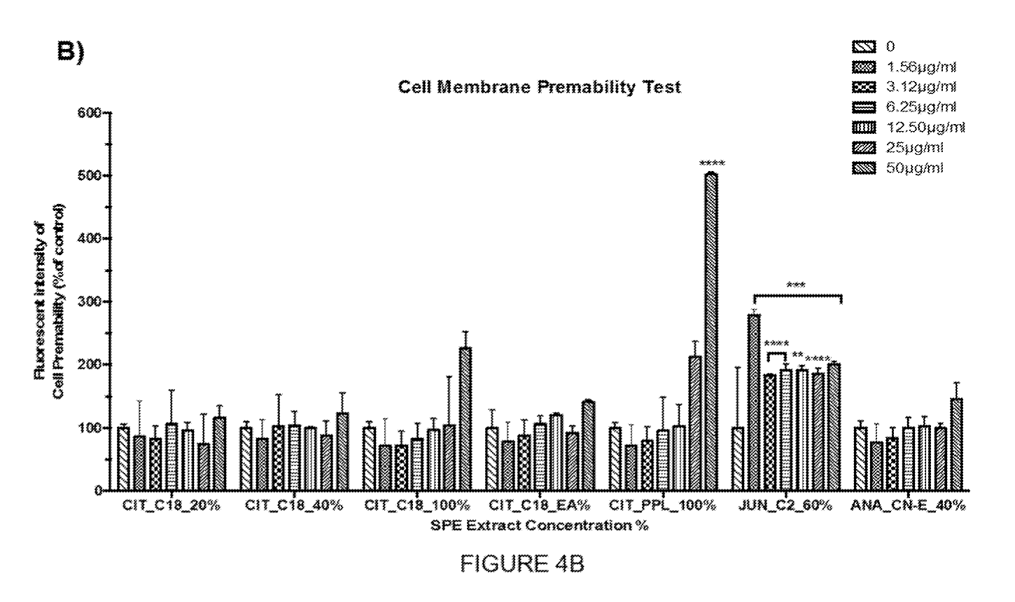

To test for mitochondrial superoxide production the inventors measured MitoSOX Red fluorescence in the mitochondrial compartment. Mitochondria play a fundamental role in apoptosis, which can be triggered by increased reactive oxygen species (ROS). MitoSOX oxidation was significantly higher in all extracts compared with control conditions. JUN_C2_60% significantly induced mitochondrial superoxide in a dose-dependent manner (see FIG. 4A). FIG. 4B shows the effect of SPE plant fractions on the membrane permeability of HeLa cells after 24 h: only CIT_PPL_100% caused a significant difference in membrane permeability and only at the highest concentration 50 □g/ml (****p<0.0001); all concentrations of JUN_C2_60% caused a significant difference, with the most significant difference evident at the lowest concentration 1.56 □g/ml (***p<0.0003).

### Characterization of Cell Apoptosis Signaling Genes: Activation of Caspase-9 and p53

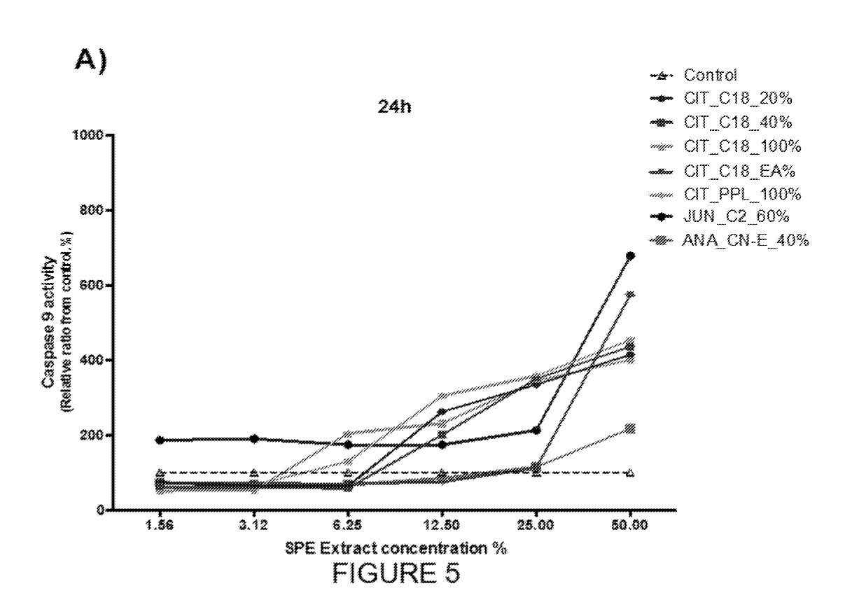

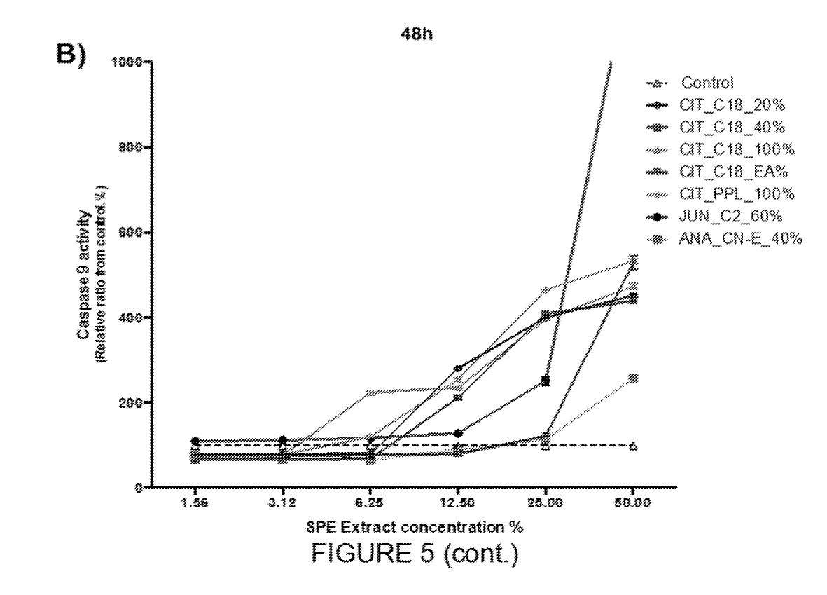

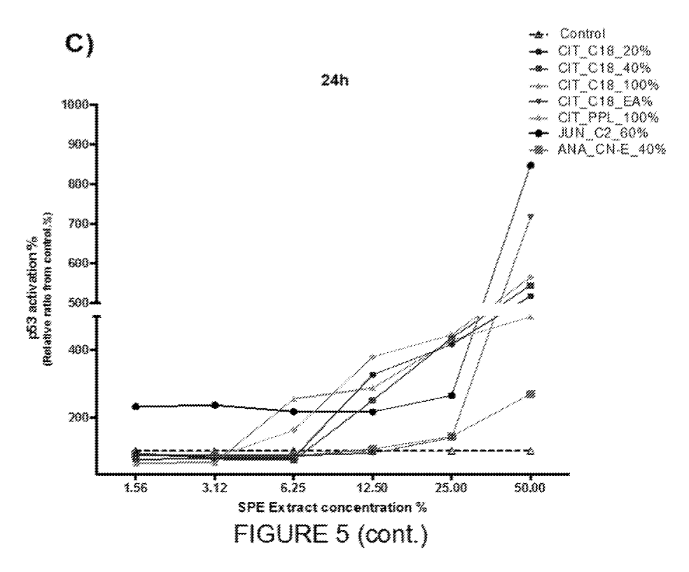

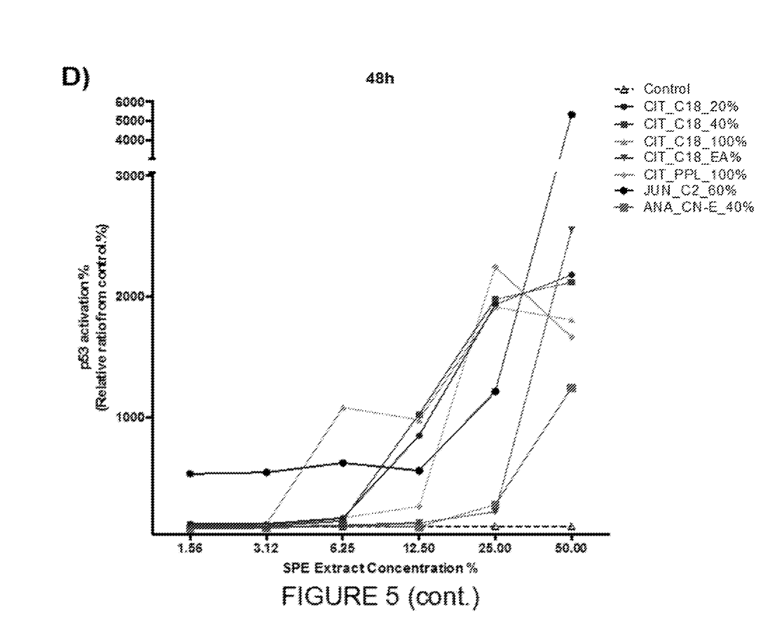

To study the molecular mechanism underlying apoptotic processes, the inventors tested cells for the activation of caspase-9 and p53 in a dose- and time-dependent manner. Caspase activation plays a vital role in the initiation and progress of apoptosis. As shown in FIG. 5, the activity of caspase-9 increases significantly at 24 h, but remained high even after 48 h of treatment, indicating an increasingly toxic effect of the extract on HeLa cells. Treatment with JUN_C2_60% showed the highest activation of both caspase-9 and p53 starting from the lowest concentration (3.12 μg/ml), indicating the activation of an apoptotic signaling pathway.

### Assessment of the Double-Strand Breaks in the DNA According to the Level of Histone H2AX Phosphorylation

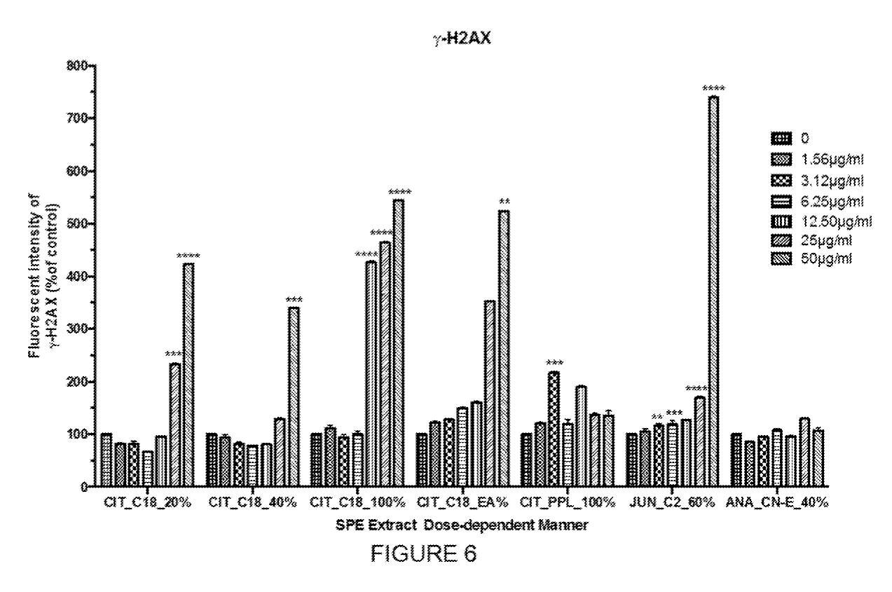

The induction phosphorylation status of Histone H2AX is considering a good cell marker to investigate genotoxcity of the SPE extract. Using HCS, we observed that when incubated with SPE fractions, double-strand breaks occurred in the DNA and histone H2AX was rapidly phosphorylated. As shown in FIG. 6, fractions from C. colocynthis induced a distinct accumulation of histone H2AX with increasing dosage: CIT_C18_20% and CIT_C18_100% had significantly more histone H2AX than did the control (CIT_C18_20%=25 μg/ml, **p<0.0002 and CIT_C18_100%=50 μg/ml, ****p<0.0001). JUN_C2_60% induced DNA damage significantly in different dose dependent manner: 3.12 μg/ml, 6.25 μg/ml, 25 μg/ml, and 50 μg/ml corresponded with **p<0.00029, **p<0.0003, ****p<0.0001, ****p<0.0001, respectively. Treatment with ANA_CN-E_40% showed no significant induction of histone H2AX.

### Chemical Analysis

To identify bioactive compounds in the JUN_C2_60% fraction extract, samples were subjected to extensive chemical analysis using GC-MS, LC-MS, and NMR. Using GC-MS the inventors found that the majority of compounds matched the mass spectra of those in the NIST library, including 2,2-dimethoxybutane, 2,6-dimethylbenzaldehyde, 3-trifluoroacetoxydodecane, 2,4-ditert-butylphenol, Ethyl 4-ethoxybenzoate, dodecyl acrylate, Stearic acid, hexanedioic acid, bis(2-ethylhexyl) ester and propanoic acid, 3,3′-thiobis-,didodecyl ester (Table 5).

TABLE 5
Phytochemicals identified in extract JUN_C2_60% by GC/MS

| S. | R. Time | | | Molecular | Molecular |
|---|---|---|---|---|---|
| No. | (min) | Proposed Name | MF | Formula | Weight |
| 1 | 4.632 | 2,2-Dimethoxybutane | 795 | C₆H₁₄O₂ | 118 |
| 2 | 12.170 | 2,6- | 750 | C₉H₁₀O | 134 |
| | | Dimethylbenzaldehyde | | | |
| 3 | 15.461 | 3-Trifluoro- | 709 | C₁₄H₂₅F₃O₂ | 282 |
| | | acetoxydodecane | | | |
| 4 | 15.904 | Phenol, 2,4-bis(1,1 | 862 | C₁₄H₂₂O | 206 |
| | | dimethylethyl)- | | | |
| 5 | 16.186 | Benzoic acid, | 648 | C₁₁H₁₄O₃ | 194 |
| | | 4-ethoxy-, ethyl ester | | | |
| 6 | 18.059 | Dodecyl acrylate | 859 | C₁₅H₂₈O₂ | 240 |
| 7 | 22.743 | Octadecanoic acid | 631 | C₁₈H₃₆O₂ | 284 |
| 8 | 24.731 | Hexanedioic acid, | 697 | C₂₂H₄₂O₄ | 370 |
| | | bis(2-ethylhexyl) | | | |
| | | ester | | | |
| 9 | 59.772 | Propanoic acid, 3,3′- | 637 | C₃₀H₅₈O₄S | 514 |
| | | thiobis-, didodecyl | | | |
| | | ester | | | |

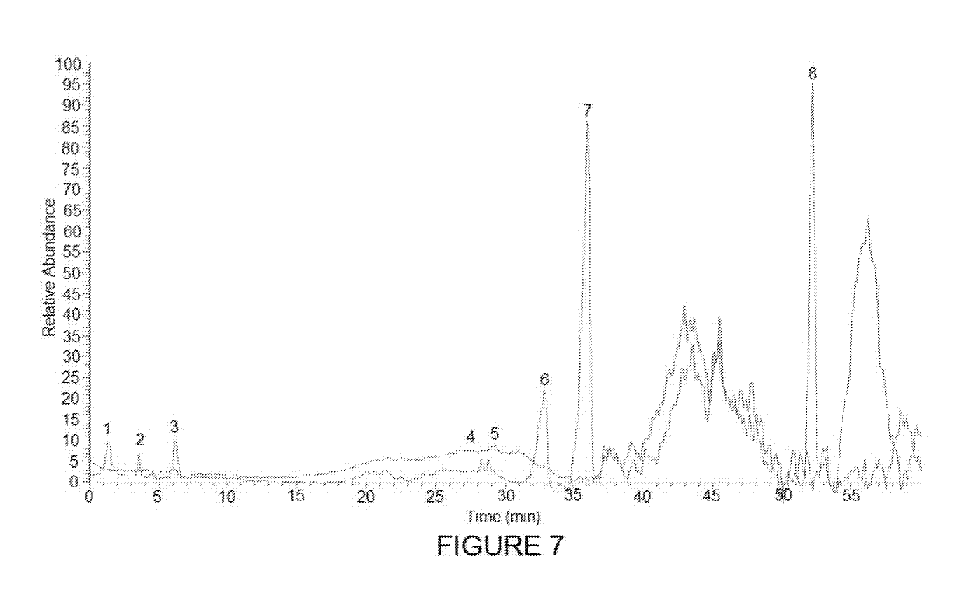

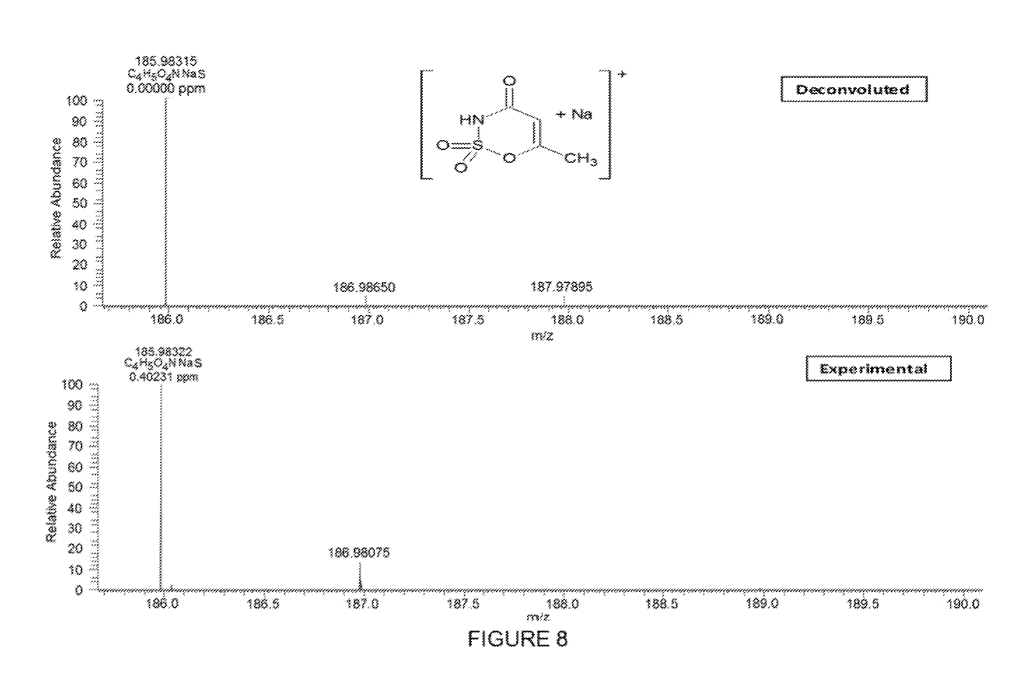

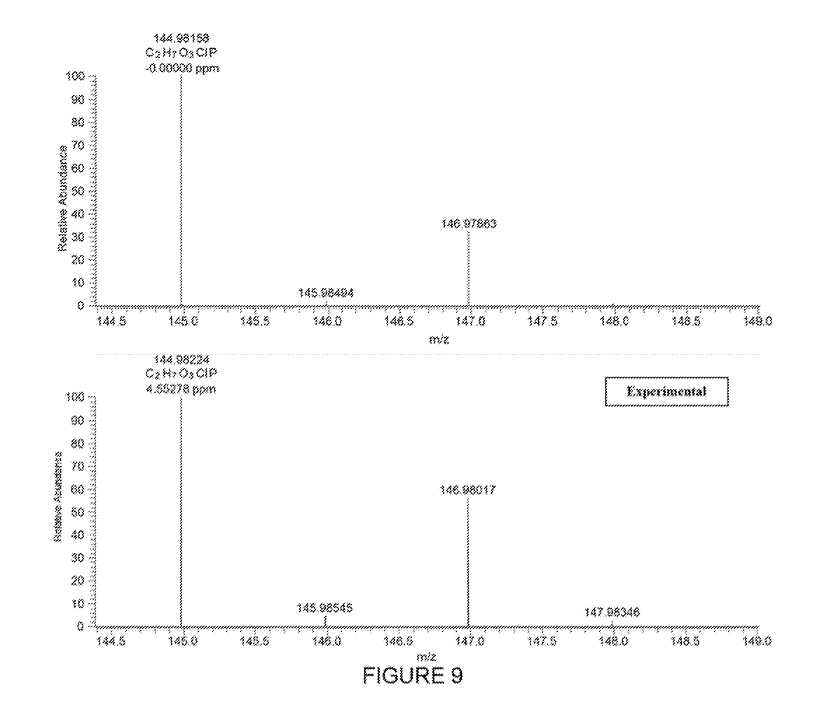

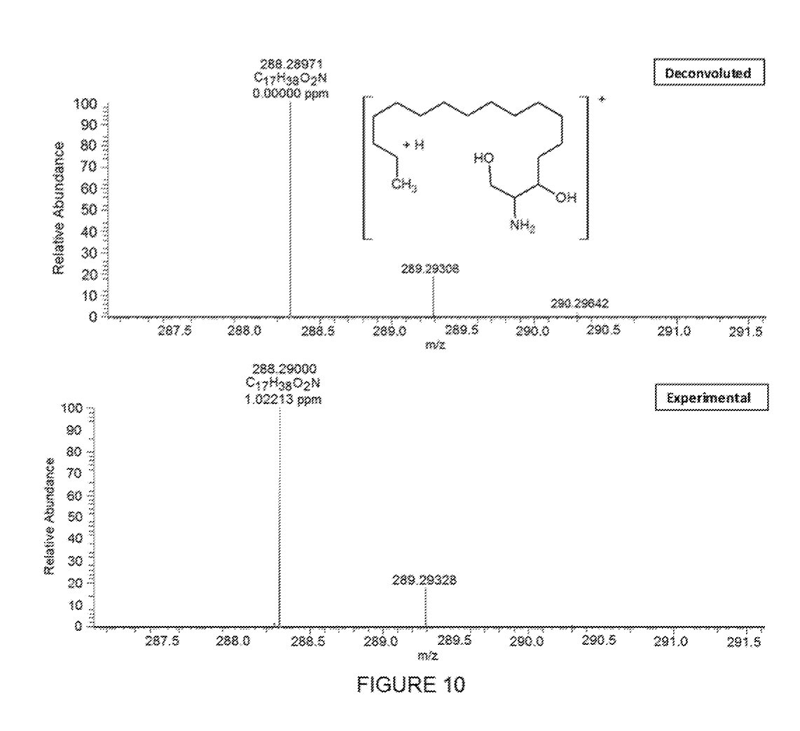

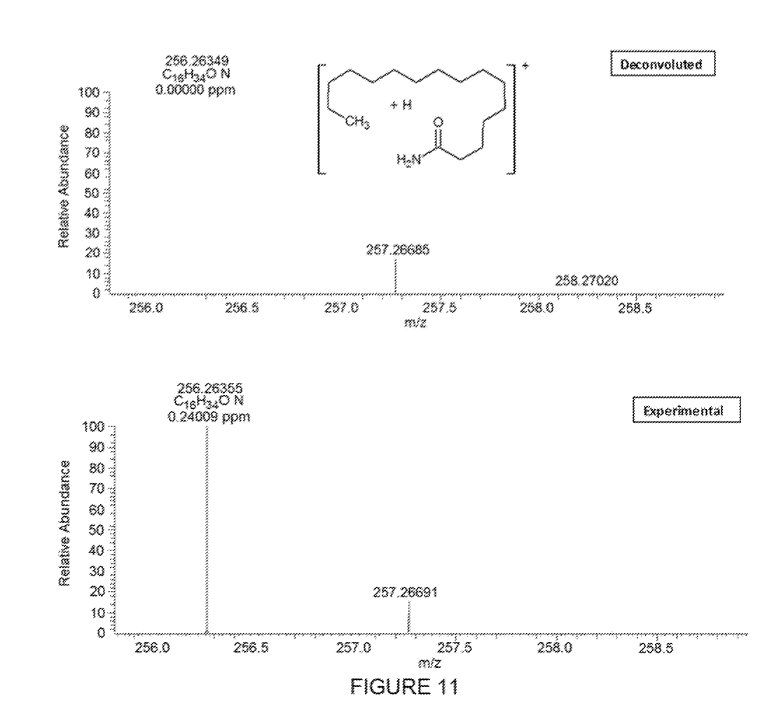

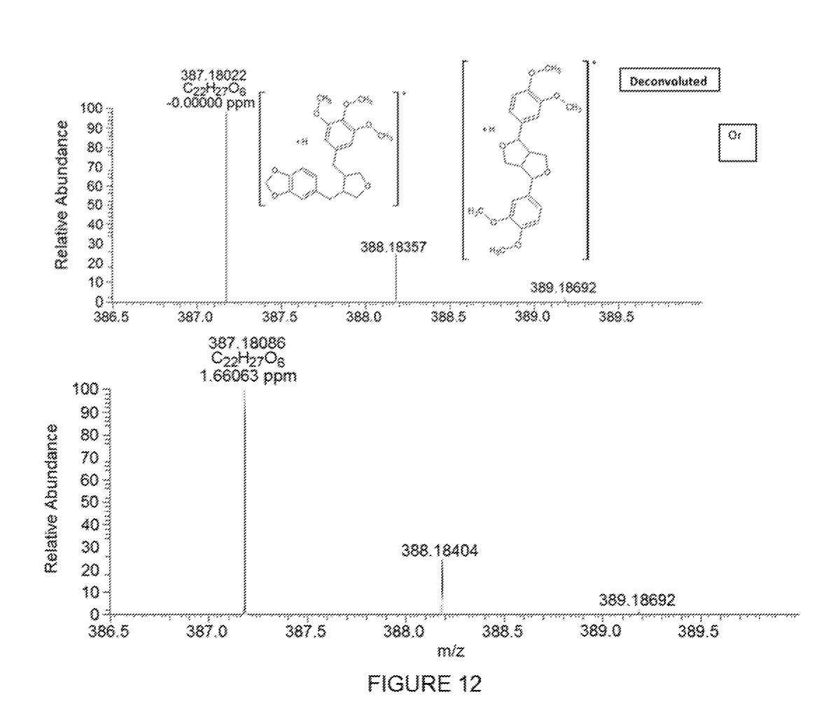

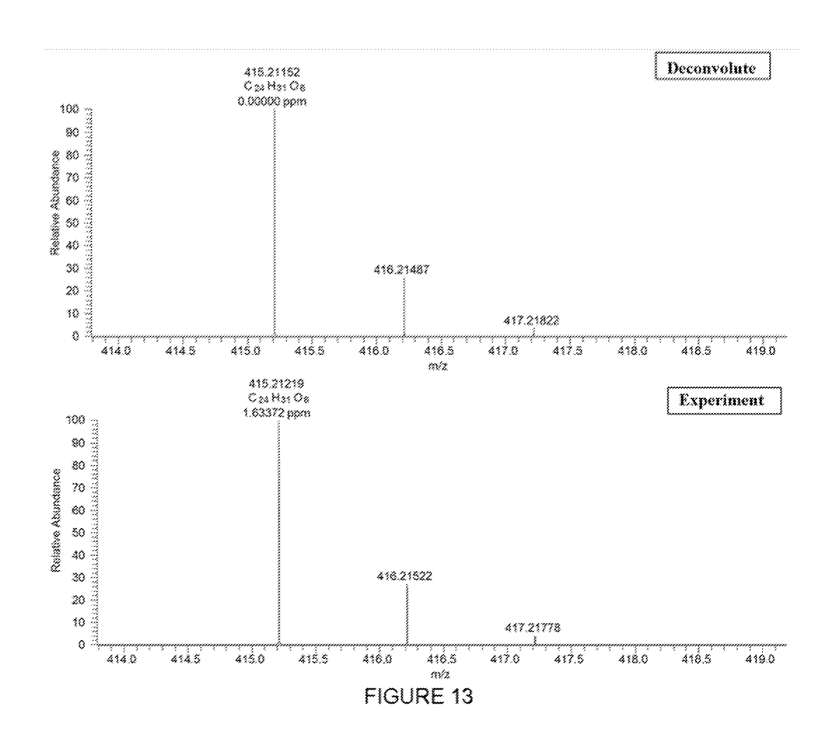

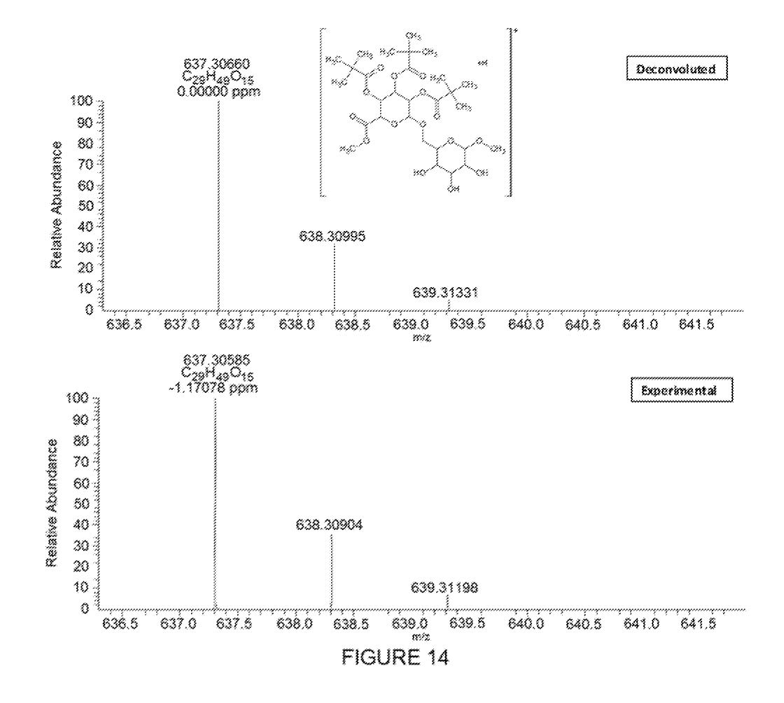

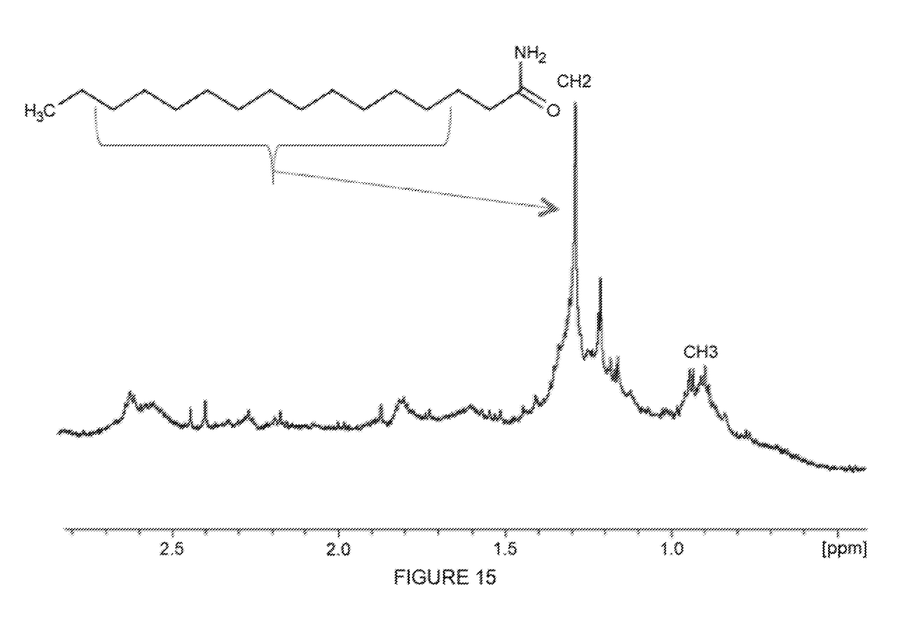

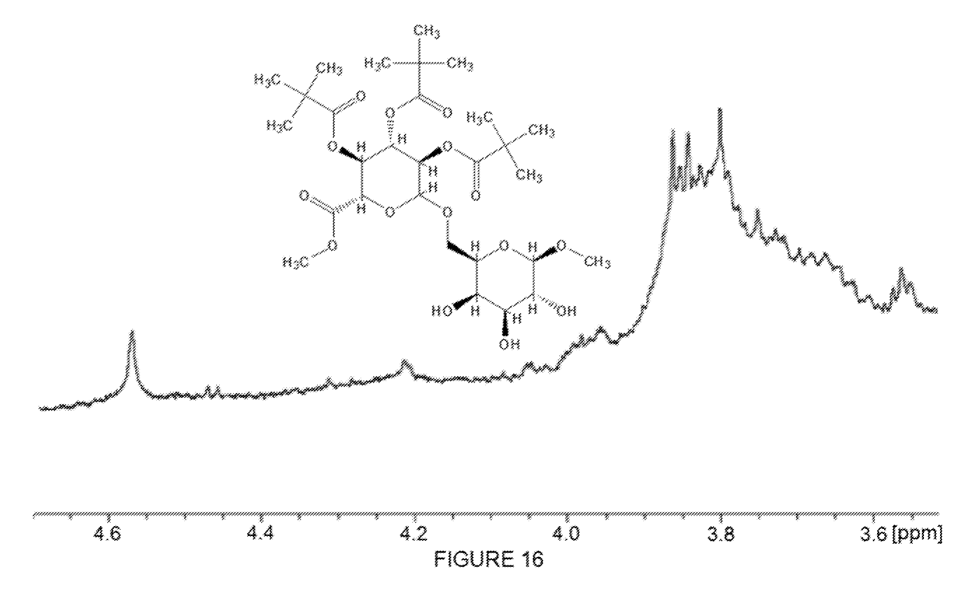

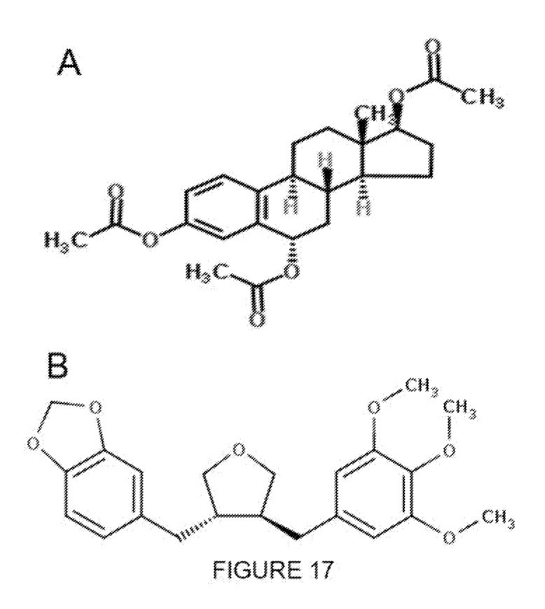

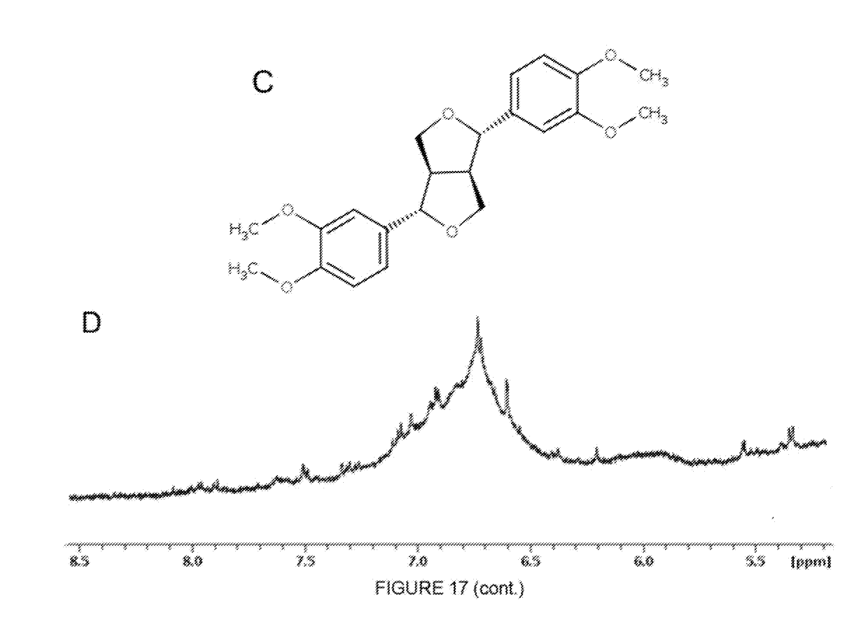

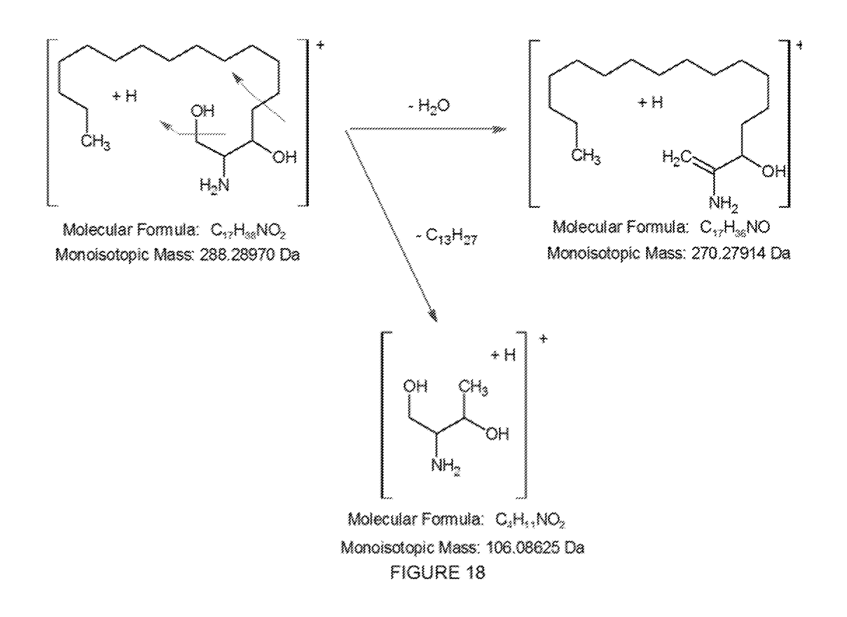

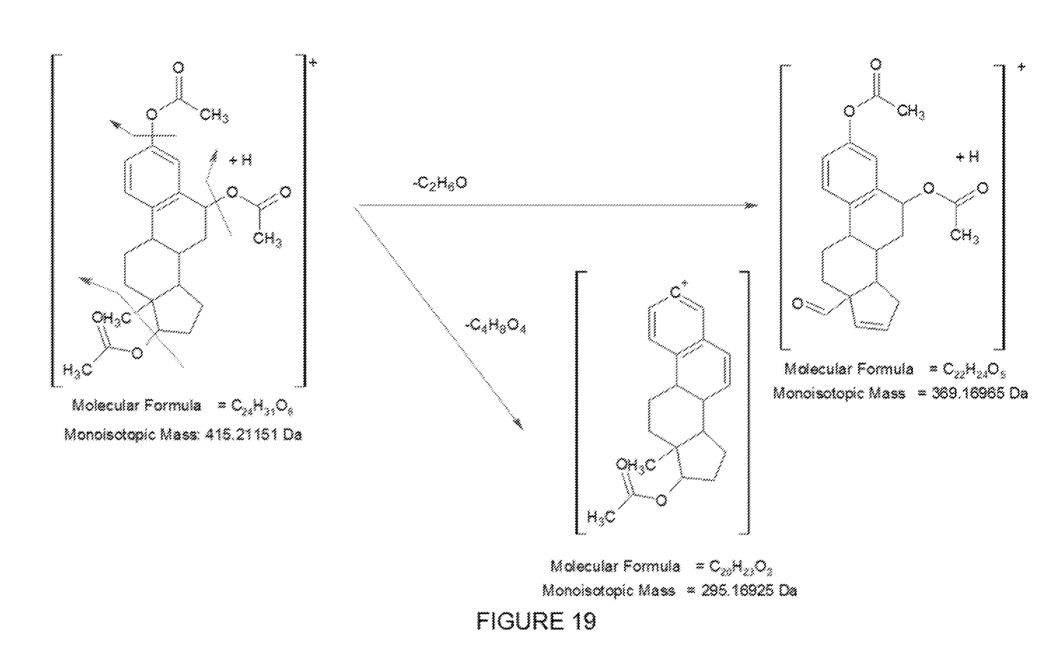

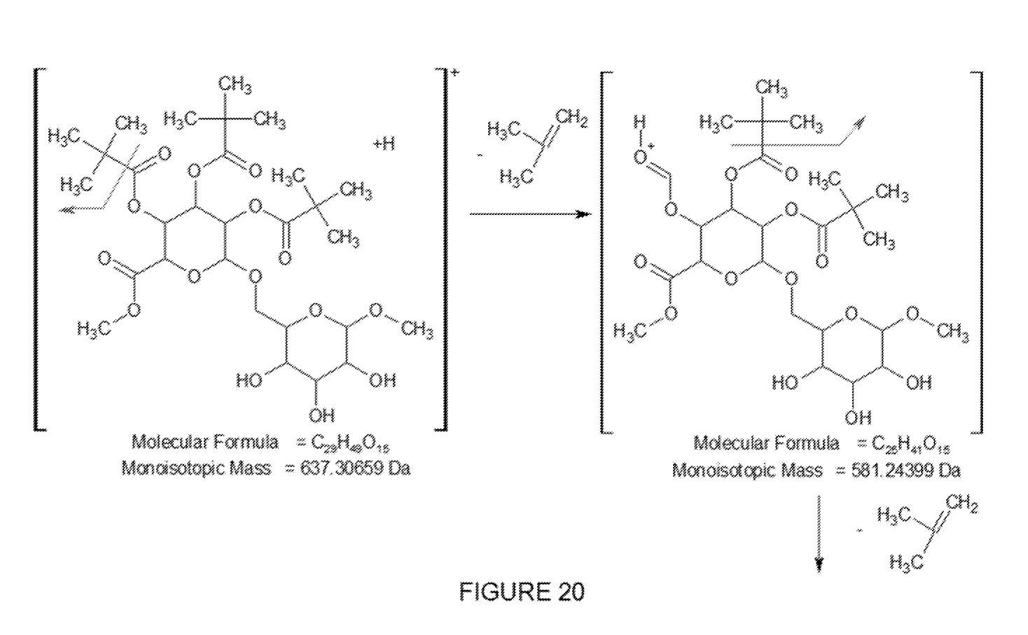

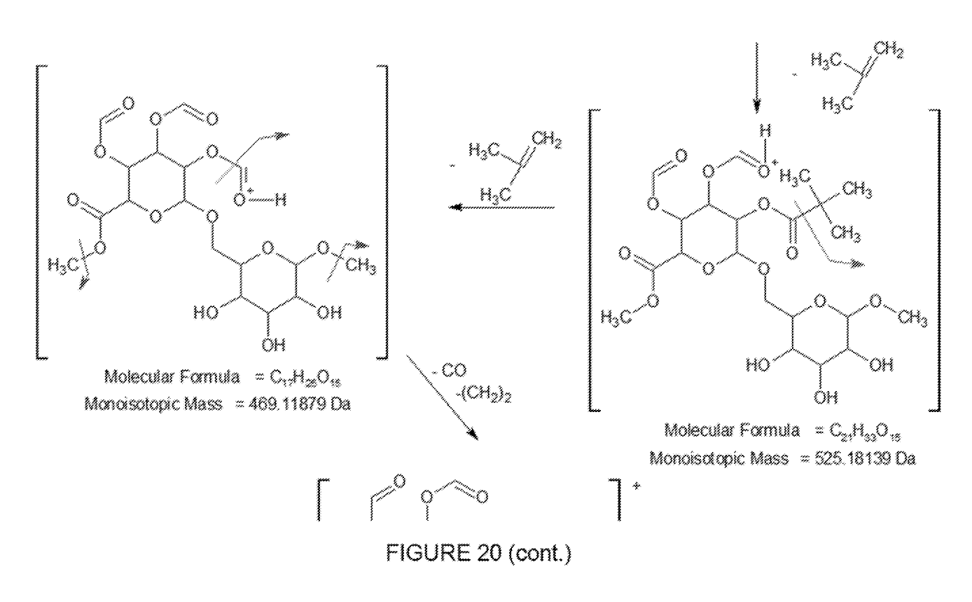

From the LC-MS chromatogram (FIG. 7), eight peaks were identified, corresponding to the following compounds: ethephon, (+)-eudesmin or burseran and sphinganine, palmitic amide, acesulfame-Na, methyl6-O-[2,3,4-tris-O-(2,2-dimethylpropanoyl)-6-methyl-β-D-glucopyranuronosyl]-βDgalactopyranoside triacetate, and estra-1,3,5(10)-triene-3,6beta,17beta-triol triacetate (FIGS. 8-14). MS/MS fragmentation studies were performed for some compounds to determine their chemical structure (FIGS. 18-20). Table 6 summarizes these compounds. Next, high resolution NMR spectroscopy was employed to record the proton NMR spectrum of the JUN_C2_60% fraction extract. A ¹H NMR spectrum was used to show different peaks in the aliphatic region, such as signals between 0.8-1 ppm, which are generally assigned as CH₃ signals, and a strong peak at 1.13 ppm, confirming that the sample contains many CH₂ groups (FIG. 15). The broad peak at 1.3 ppm is usually rated to lipid or lipid-like molecules with several adjacent (CH₂) groups, which supports observations from MS proposed molecules of the presence of analytes 4 and 5. The spectrum also shows broad peaks in the region of sugar and OH signal regions (FIG. 16). Moreover, The ¹H NMR spectra support results from the MS for the presence of aromatic molecules, such as analyte 6 and analyte 7, as summarized in FIG. 17.

TABLE 6
Proposed identified chemicals from JUN_C2_60% by LC/MS.

| Chromatogram | Molecular | Accurate Masse | | Chemical | | |
|---|---|---|---|---|---|---|
| Peaks | Ion | (Δm ≤ 5 ppm) | Proposed Name | Structure | Software | MS/MS (n) |
| 1 | [M₁ + Na]⁺ | 185.98322 | Acesulfame-Na | C₄H₅NO₄SNa | Metlin | NA |
| 2 and 3 | [M_{2/3} + H]⁺ | 144.98158 | Ethephon | C₂H₆ClO₃P | Metlin | NA |
| 4 | [M₄ + H]⁺ | 288.29000 | C17 Sphinganine | C₁₇H₃₇NO₂ | Metlin | MS/MS (2) |
| | | | (heptadecasphinganine) | | | |
| 5 | [M₅ + H]⁺ | 256.26355 | Palmitic amide | C₁₆H₃₃NO | Metlin | NA |
| 6 | [M₆ + H]⁺ | 387.18086 | Burseran | C₂₂H₂₆O₆ | Metlin | NA |
| | | | (+)Eudesmin | C₂₂H₂₆O₆ | Metlin | NA |
| 7 | [M₇ + H]⁺ | 415.21219 | Estra-1,3,5(10)-triene-3,6beta,17beta- | C₂₄H₃₀O₆ | Metlin | MS/MS (2) |
| | | | triol triacetate | | | |
| 8 | [M₈ + H]⁺ | 637.30585 | Methyl 6-O-[2,3,4-tris-O-(2,2- | C₂₉H₄₈O₁₅ | Met-Frag | MS/MS (5) |
| | | | dimethylpropanoyl)-6-methyl-β-D- | | | |
| | | | glucopyranuronosyl]-β-D- | | | |
| | | | galactopyranoside | | | |

### Discussion

### Using High-Content Screening as a Tool for Natural Products Drug Discovery

This study emphasizes the strength of cytological profiling for natural products drug discovery using High-Content Screening (HCS). In recent years, HCS has developed from a promising concept into an efficient methodology and indispensable tool. HCS now can be implemented into early drug discovery process as a result of its recent technological advances (Rausch, 2006; Young et al., 2008). The chemical composition, mode of action, and toxicity of Saudi Arabian plants with medicinal properties are not well understood (El-Ghazali et al., 2010).

### Target Prediction Using HCS

Here, the inventors emphasize the strength of cytological profiling to characterize these properties, in three native Saudi Arabian plants: C. colocynthis, J. phoenicea, and A. hierochuntica.

A library of known small molecules with assigned modes of action was used as reference compounds and resulting cytological profiles were compared to profiles retrieved from fractionated plant extracts. Prediction of MOAs by comparing similarities of HCS-derived phenotypic profiles was successfully implemented for pure compounds as well as fractionated extracts (R. Voolstra, 2016; Schulze et al., 2013; Young et al., 2008). Cluster analysis of the cytological profiles revealed high similarities between seven extracted fractions and topo II inhibitors. Because topoisomerase enzymes are among the primary targets of chemotherapy treatment, we pursued additional experiments to more specifically evaluate the effect of these seven fractions on cancer cells. We found that five extracted fractions of C. colocynthis, a plant found abundantly in Saudi Arabia, were phenotypically similar to topo II inhibitors; for example, the anticancer drugs etoposide and camptothecin, which induce topo II formation (Li and Liu, 2001; Montecucco and Biamonti, 2007) and activates several molecules, such as histone H2AX, p53, ATM, and Chk1/2, that trigger responses to DNA damage (Tanaka et al., 2007; Wu et al., 2014).

### Target Validation for Target Prediction

The data confirmed that the extracted fractions had cytotoxic effects on HeLa cells, sharply decreasing cell numbers in a dose- and time-dependent manner (FIG. 3A-B). We then studied the effect of the extracted fractions on mitochondrial stress, cell membrane permeability, and cell cycle arrest. Mitochondrial superoxide indicator was detected by MitoSOX Red fluorigenic dye. MitoSOX fluorescence localizes to the mitochondrion due to its hydrophobic nature and its positively charged triphenylphosphonium moiety (Mukhopadhyay et al., 2007; Piacenza et al., 2007) Previous research has shown that oxidative stress due to an increased number of reactive oxygen species is a signature of selectivity for tumor toxicity (Rashad et al., 2011; Wu et al., 2014). We found that with the JUN_C2_60% treatment, tumor toxicity increased most significantly and permeability caused to the cell membrane led to cell death.

Because cell cycle dysfunction is a characteristic of cancerous cells, a natural product that is capable of blocking the cell cycle could be considered a potential anticancer compound (Williams and Stoeber, 2012). Our findings showed that fractions of C. colocynthis and ANA_CN-E_40% caused strong arrest in the G0/G1 phase while JUN_C2_60% caused cell cycle arrest in the G2/M phase (FIG. 3C). Although topoisomerase inhibitors typically show similar cytotoxic affects, they have a different affect on the cell cycle (Tanaka et al., 2007).

Topoisomerase forms double-strand breaks (DSBs) in DNA that are necessary to unwind it for repairs; however, if the strands are not reconnected it can lead to cell death. Therefore, when topo II inhibitor drugs form a complex between topoisomerase and DNA, DNA goes unrepaired, resulting in apoptosis (Ichijima et al., 2005; Negritto, 2010; Sordet et al., 2003). The formation of DSBs during the DNA replication process correlates well with an initial increase in γ-H2AX, which is considered to be a marker for stalled and collapsed replication forks and reduced activities of topoisomerases I and II. Therefore, H2AX levels are an important sensitivity marker of DNA damage processes and acts as a marker for double-strand break formation (Bonner et al., 2008; Ichijima et al., 2005; Mah et al., 2010; McManus and Hendzel, 2005; Podhorecka et al., 2010). In addition, increases in histone H2AX phosphorylation have been observed to correlate with cell cycle arrest and the activation of apoptotic signaling pathways, such as caspase-9 and P53 (Azarova et al., 2007; Bailly, 2012; Huang et al., 2003). We evaluated the abundance of phosphorylated H2AX as an indicator of DNA damage to find that all treatments with C. colocynthis and the JUN_C2_60% treatment had significantly high levels of phosphorylation, indicative of DNA damage (see FIG. 6).

The results also showed that treatment of HeLa cells with all tested concentrations of C. colocynthis and the JUN_C2_60% treatment caused increased caspase-9 and the p53 tumor-suppressor protein activities (see FIG. 5) Although p53 has several roles in regulating the cell cycle, its overexpression is associated with obstructing cell grown and inducing apoptosis at the G0/G1 cell cycle checkpoint(Wieler et al., 2003; Wolff et al., 2008; Yin et al., 2011). Moreover, activation of caspase is integral to apoptosis (Hail Jr, 2005). Thus the upregulation of these two proteins is indicative of DNA damage.

### Chemical Analysis of the Active Fraction JUN_C2_60%

Particularly high levels of γH2AX expression in cells with a G2/M-phase DNA content suggests that JUN_C2_60% fraction may be a potential topo inhibitor. Because JUN_C2_60% also contributed to the apoptotic pathway, the inventors subjected it to chemical profiling to find several structures in common with compounds known to have medicinal value.

The 2,2-dimethoxybutane found in the JUN_C2_60% fraction was known to be toxic to microbial membranes, but has never before been shown to behave as an anticancer compound (Kalt and Cock, 2014; Sani et al., 2015; Sikkema et al., 1995). 2,4-ditert-butylphenol is a phenolic compound and secondary metabolite in plant. It has been reported to possess strong antioxidant activity, anticancer activity, antifungal activity and antibacterial (Dharni et al., 2014; Malek et al., 2009; Sani et al., 2015; Yoon et al., 2006). It shows cytotoxic effects against MCF7 cells with an IC50 value of 5.75 μg/mL, KB cells (IC50 0.81 μg/mL) and CasKi cells (IC50 4.5 μg/mL). Dhami et al., studied the antifungal effect of this compound and conclude that it potentially binds to β-tubulin in microtubules, inhibiting eukaryotic cell growth by destroying their dynamic instability as effecting cytoskeletal polymers in eukaryotic cells (Dharni et al., 2014). This may suggest JUN_C₂_60% fraction could have an effect on β-tubulin. From the HCS panel, these show high intensity effects on tubulin which require further study using different concentration of fraction in order to understand the exact effect on β-tubulin microtubule (FIG. 4). 3-Trifluoroacetoxydodecane has been reported to have anticancer and antimicrobial activities (SUDHA et al.) Stearic acid, a saturated long-chain fatty acid with an 18-carbon backbone is found in various animal and plant fats, and is a major component of cocoa butter and shea butter. Stearic acid has been reported to exert anti-cancer as well as anti-inflammatory effects (Agoramoorthy et al., 2007; Seidel and Taylor, 2004). It has been reported that stearic acid inhibited human cervical cancer (HOG-1) cell growth and DNA synthesis in a different concentrations. It has been show it effects on early signals leading to cell proliferation (Beesley et al., 1993). This correspond will with our finding, J. phoenicea extract shows inhibition in cell growth on G2/M phase and detect high level of γH2AX expression which use as marker for DNA damage. Stearic acid was previously found in J. phoenicea from Southern Tunisia (Ben Ali et al., 2015). Propanoic acid, 3,3′-thiobis-,didodecyl ester has been reported for potential anti-inflammatory action[70]. Ethyl 4-ethoxybenzoate has been reported as natural local anesthetics (Quan et al., 1996). Of the compounds that the screening detected, dodecyl acrylate, 2,6-Dimethylbenzaldehyde and I-Iexanedioic acid,bis(2-ethylhexyl) ester have had no reported anticancer activity unlike ethephon, (+)-eudesmin or burseran and sphinganine (AHN and SCHROEDER, 2006; Duke, 1986; Messina et al., 2015; Yurdakok et al., 2014). Sphinganine is reported as an anticancer compound, which support the present results due to its presence is associated with activation of p38 MAPK and JNK, and weak inhibition of AKT, which explains the DNA damage and activation of p53 by J. phoenicea fraction (AHN and SCHROEDER, 2006). Using LC-MS, the inventors found Methyl6-O[2,3,4-tris-O-(2,2-dimethylpropanoyl)-6-methyl-β-D-glucopyranuronosyl]-βDgalactopyranoside triacetate and Estra-1,3,5(10)-triene-3,6beta,17beta-triol triacetate. Estra-1,3,5(10)-triene-3,6beta,17beta-triol triacetate is used as anti-inflammatory drug but no activity were reported for Methyl6-O-[2,3,4-tris-O-(2,2-dimethylpropanoyl)-6-methyl-β-D-glucopyranuronosyl]-β-Dgalactopyranoside triacetate compound.

### CONCLUSIONS

The analysis presented herein reinforces the power of HCS for drug discovery. HCS allows reassessing historically selected medicinal plants for identification of bioactive compounds. Cytological profiles allowed us to identify topoisomerase II inhibitor activity in the plant fractions tested, verifying the anticancer compounds in the plant. Further studies are needed to evaluate and better characterized the active compounds identified in our study. This can be done by guided fractionation using HPLC to isolate and eliminate the active from the non-active compounds and re-test them on the system. Furthermore, synthesize the active compounds and test them on vivo system.

### REFERENCES (FIRST SET)

Aggarwal, B. B., Danda, D., Gupta, S., Gehlot, P., 2009. Models for prevention and treatment of cancer: problems vs promises. Biochemical pharmacology 78(9), 1083-1094.

Agoramoorthy, G., Chandrasekaran, M., Venkatesalu, V., Hsu, M., 2007. Antibacterial and antifungal activities of fatty acid methyl esters of the blind-your-eye mangrove from India. Brazilian Journal of Microbiology 38(4), 739-742.

AHN, E. H., SCHROEDER, J. J., 2006. Sphinganine causes early activation of JNK and p38 MAPK and inhibition of AKT activation in HT-29 human colon cancer cells. Anticancer research 26(1A), 121-127.

Al-Talla, Z., Akrawi, S., Emwas, A., 2011. Solid state NMR and bioequivalence comparison of the pharmacokinetic parameters of two formulations of clindamycin. International journal of clinical pharmacology and therapeutics 49(7), 469-476.

Alfarhan, A. H., Chaudhary, S. A., Thomas, J., 1998. Notes on the flora of Saudi Arabia. Journal of King Saud University.

Azarova, A. M., Lyu, Y. L., Lin, C. -P., Tsai, Y. -C., Lau, J. Y. -N., Wang, J. C., Liu, L. F., 2007. Roles of DNA topoisomerase II isozymes in chemotherapy and secondary malignancies. Proceedings of the National Academy of Sciences 104(26), 11014-11019.

Bailly, C., 2012. Contemporary challenges in the design of topoisomerase II inhibitors for cancer chemotherapy. Chemical reviews 112(7), 3611-3640.

Beesley, J., Soutter, W., White, J., 1993. Effect of stearic acid on human cervical cancer cell growth, Eicosanoids and Other Bioactive Lipids in Cancer, Inflammation and Radiation Injury. Springer, pp. 573-575.

Ben Ali, M. J., Guesmi, F., Harrath, A. H., Alwasel, S., Hedfi, A., Ncib, S., Landoulsi, A., Aldahmash, B., Ben-Attia, M., 2015. Investigation of Antiulcer and Antioxidant Activity of Juniperus phoenicea L. (1753) Essential Oil in an Experimental Rat Model. Biological and Pharmaceutical Bulletin 38(11), 1738-1746.

Bonner, W. M., Redon, C. E., Dickey, J. S., Nakamura, A. J., Sedelnikova, O. A., Solier, S., Pommier, Y., 2008. γH2AX and cancer. Nature Reviews Cancer 8(12), 957-967.

Cairnes, D. A., Ekundayo, O., Kingston, D. G., 1980. Plant anticancer agents. X. Lignans from Juniperus phoenicea. Journal of natural products 43(4), 495-497. Cancer, I.A.f.R.o., 2014. World cancer report 2014. Geneva: WHO.

Cragg, G. M., Newman, D. J., 2004. A tale of two tumor targets: topoisomerase I and tubulin. The Wall and Wani contribution to cancer chemotherapy. Journal of Natural Products 67(2), 232-244.

Dharni, S., Maurya, A., Samad, A., Srivastava, S. K., Sharma, A., Patra, D.D., 2014. Purification, characterization, and in vitro activity of 2, 4-di-tert-butylphenol from Pseudomonas monteilii PsF84: conformational and molecular docking studies. Journal of agricultural and food chemistry 62(26), 6138-6146.

Diercks, T., Coles, M., Kessler, H., 2001. Applications of NMR in drug discovery. Current opinion in chemical biology 5(3), 285-291.

Duke, J. A., 1986. Folk anticancer plants containing antitumor compounds, Plants in Indigenous Medicine and Diet—Biobehavioral Approaches. Redgrave New York, pp. 70-90.

Eklund, E. A., Gabig, T., 1990. Purification and characterization of a lipid thiobis ester from human neutrophil cytosol that reversibly deactivates the O2—generating NADPH oxidase. Journal of Biological Chemistry 265(15), 8426-8430.

El-Ghazali, G. E., Al-Khalifa, K. S., Saleem, G. A., Abdallah, E. M., 2010. Traditional medicinal plants indigenous to Al-Rass province, Saudi Arabia. J. Med. Plants Res 4(24), 2680-2683.

Emwas, A. -H. M., Al-Talla, Z. A., Kharbatia, N. M., 2015a. Sample Collection and Preparation of Biofluids and Extracts for Gas Chromatography—Mass Spectrometry. Metabonomics: Methods and Protocols, 75-90.

Emwas, A. -H. M., Al-Talla, Z. A., Yang, Y., Kharbatia, N. M., 2015b. Gas Chromatography—Mass Spectrometry of Biofluids and Extracts. Metabonomics: Methods and Protocols, 91-112.

Ge, Q., Zhang, A. Q. Sun, P. L., 2010. Isolation, Purification and Structureal Characterization of a Novel Water-Soluble Glucan From the Fruiting Bodies of Phellinus BAUMH PILÂT, Journal of Food Biochemistry 34(6), 1205-1215.

Ghosh, R. N., Grove, L., Lapets, O., 2004. A quantitative cell-based high-content screening assay for the epidermal growth factor receptor-specific activation of mitogen-activated protein kinase. Assay and drug development technologies 2(5), 473-481.

Giuliano, K. A., DeBiasio, R. L., Dunlay, R. T., Gough, A., Volosky, J. M., Zock, J., Pavlakis, G. N., Taylor, D. L., 1997. High-content screening: a new approach to easing key bottlenecks in the drug discovery process. Journal of Biomolecular Screening 2(4), 249-259.

Hail Jr, N., 2005. Mitochondria: a novel target for the chemoprevention of cancer. Apoptosis 10(4), 687-705.

Haney, S. A., LaPan, P., Pan, J., Zhang, J., 2006. High-content screening moves to the front of the line. Drug discovery today 11(19), 889-894.

Huang, X., Traganos, F., Darzynkiewicz, Z., 2003. DNA Damage Induced by DNA Topoisomerase I-and Topoisomerase II-Inhibitors Detected by Histone H2AXphosphorylation in Relation to the Cell Cycle Phase and Apoptosis. Cell Cycle 2(6), 613-618.

Hussain, A. I., Rathore, H. A., Sattar, M. Z., Chatha, S. A., Sarker, S. D., Gilani, A. H., 2014. Citrullus colocynthis (L.) Schrad (bitter apple fruit): A review of its phytochemistry, pharmacology, traditional uses and nutritional potential. Journal of ethnopharmacology 155(1), 54-66.

Ichijima, Y., Sakasai, R., Okita, N., Asahina, K., Mizutani, S., Teraoka, H., 2005. Phosphorylation of histone H2AX at M phase in human cells without DNA damage response. Biochemical and biophysical research communications 336(3), 807-812.

Jackson, S. P., Bartek, J., 2009. The DNA-damage response in human biology and disease. Nature 461(7267), 1071-1078.

Kalt, F., Cock, I. E., 2014. Gas chromatography-mass spectroscopy analysis of bioactive petalostigma extracts: Toxicity, antibacterial and antiviral activities. Pharmacognosy magazine 10 (Suppl 1), S37.

Keifer, P. A., 2000. NMR spectroscopy in drug discovery: tools for combinatorial chemistry, natural products, and metabolism research, Progress in drug research. Springer, pp. 137-211.

Khorshid, F., Emwas, A. -H., Mahboub, F., 2015. The Cytotoxic Effect of Small and Large Molecules of PMF Fraction Extracted from Camel Urine on Cancer Cells. British Journal of Medicine and Medical Research.

Korn, K., Krausz, E., 2007. Cell-based high-content screening of small-molecule libraries. Current opinion in chemical biology 11(5), 503-510.

Kuete, V., Efferth, T., 2011. Pharmacogenomics of Cameroonian traditional herbal medicine for cancer therapy. Journal of Ethnopharmacology 137(1), 752-766.

Li, T. -K., Liu, L. F., 2001. Tumor cell death induced by topoisomerase-targeting drugs. Annual review of pharmacology and toxicology 41(1), 53-77.

Liu, L. F., 1989. DNA topoisomerase poisons as antitumor drugs. Annual review of biochemistry 58(1), 351-375.

Mah, L., El-Osta, A., Karagiannis, T., 2010. δH2AX: a sensitive molecular marker of DNA damage and repair. Leukemia 24(4), 679-686.

Malek, S. N. A., Shin, S. K., Wahab, N. A., Yaacob, H., 2009. Cytotoxic components of Pereskia bleo (Kunth) DC.(Cactaceae) leaves. Molecules 14(5), 1713-1724.

McManus, K. J., Hendzel, M. J., 2005. ATM-dependent DNA damage-independent mitotic phosphorylation of H2AX in normally growing mammalian cells. Molecular biology of the cell 16(10), 5013-5025.

Messina, F., Curini, M., Di Sano, C., Zadra, C., Gigliarelli, G., Rascón-Valenzuela, L. A., Robles Zepeda, R. n. E., Marcotullio, M. C., 2015. Diterpenoids and Triterpenoids from the Resin of Bursera microphylla and Their Cytotoxic Activity. Journal of natural products.

Montecucco, A., Biamonti, G., 2007. Cellular response to etoposide treatment. Cancer letters 252(1), 9-18.

Mukhopadhyay, P., Rajesh, M., Yoshihiro, K., Haskó, G., Pacher, P., 2007. Simple quantitative detection of mitochondrial superoxide production in live cells. Biochemical and biophysical research communications 358(1), 203-208.

Negritto, C., 2010. Repairing double-strand DNA breaks. Nat Educ 3(9), 26.

Nichols, A., 2006. High content screening as a screening tool in drug discovery, High Content Screening. Springer, pp. 379-387.

Nitiss, J. L., 2009. Targeting DNA topoisomerase II in cancer chemotherapy. Nature Reviews Cancer 9(5), 338-350.

Piacenza, L., Irigoin, F., Alvarez, M. N., Peluffo, G., Taylor, M. C., Kelly, J. M., Wilkinson, S. R., Radi, R., 2007. Mitochondrial superoxide radicals mediate programmed cell death in Trypanosoma cruzi: cytoprotective action of mitochondrial iron superoxide dismutase overexpression. Biochem J 403(2), 323-334.

Podhorecka, M., Skladanowski, A., Bozko, P., 2010. H2AX phosphorylation: its role in DNA damage response and cancer therapy. Journal of nucleic acids 2010.

Pommier, Y., Schwartz, R. E., Zwelling, L. A., Kohn, K. W., 1985. Effects of DNA intercalating agents on topoisomerase II induced DNA strand cleavage in isolated mammalian cell nuclei. Biochemistry 24(23), 6406-6410.

Quan, C., Mok, W. M., Wang, G. K., 1996. Use-dependent inhibition of Na+ currents by benzocaine homologs. Biophysical journal 70(1), 194.

R. Voolstra, S. K. a. C., 2016. High-resolution phenotypic profiling of natural products-induced effects on the single-cell level

Rashad, A. E., Mahmoud, A. E., Ali, M. M., 2011. Synthesis and anticancer effects of some novel pyrazolo [3,4-d] pyrimidine derivatives by generating reactive oxygen species in human breast adenocarcinoma cells. European journal of medicinal chemistry 46(4), 1019-1026.

Rausch, O., 2006. High content cellular screening. Current opinion in chemical biology 10(4), 316-320.

Saeed, A., Sharov, V., White, J., Li, J., Liang, W., Bhagabati, N., Braisted, J., Klapa, M., Currier, T., Thiagarajan, M., 2003. TM4: a free, open-source system for microarray data management and analysis. Biotechniques 34(2), 374.

Sani, H. L., Malami, I., Hassan, S. W., Muhammad, A., Alhassan, M. E. H., Muhammad, A., 2015. Effects of standardized stem bark extract of Mangifera indica L. in wistar rats with 2,4-dinitrophenylhydrazine-induced haemolytic anaemia. Pharmacognosy Journal 7(2), 89-96.

Schulze, C. J., Bray, W. M., Woerhmann, M. H., Stuart, J., Lokey, R. S., Linington, R. G., 2013. “Function-first” lead discovery: mode of action profiling of natural product libraries using image-based screening. Chemistry & biology 20(2), 285-295.

Seidel, V., Taylor, P. W., 2004. In vitro activity of extracts and constituents of Pelagonium against rapidly growing mycobacteria. International journal of antimicrobial agents 23(6), 613-619.

Sikkema, J., De Bont, J., Poolman, B., 1995. Mechanisms of membrane toxicity of hydrocarbons. Microbiological reviews 59(2), 201-222.

Sordet, O., Khan, Q. A., Kohn, K. W., Pommier, Y., 2003. Apoptosis induced by topoisomerase inhibitors. Current Medicinal Chemistry-Anti-Cancer Agents 3(4), 271-290.

SUDHA, T., CHIDAMBARAMPILLAI, S., MOHAN, V., GC-MS ANALYSIS OF BIOACTIVE COMPONENTS OF AERIAL PARTS OF KIRGANELIA RETICULATA POIR (EUPHORBIACEAE).

Tanaka, T., Halicka, H. D., Traganos, F., Seiter, K., Darzynkiewicz, Z., 2007. Induction of ATM activation, histone H2AX phosphorylation and apoptosis by etoposide: relation to cell cycle phase. Cell Cycle 6(3), 371-376.

Tannin-Spitz, T., Grossman, S., Dovrat, S., Gottlieb, H. E., Bergman, M., 2007. Growth inhibitory activity of cucurbitacin glucosides isolated from Citrullus colocynthis on human breast cancer cells. Biochemical pharmacology 73(1), 56-67.

Tavakoli, J., Miar, S., Zadehzare, M. M., Akbari, H., 2012. Evaluation of Effectiveness of Herbal Medication in Cancer Care: A Review Study. Iranian journal of cancer prevention 5(3), 144.

Taylor, D. L., 2006. Past, present, and future of high content screening and the field of cellomics, High Content Screening. Springer, pp. 3-18.

Urban, S., 2006. LC-NMR: profiling and dereplication of natural product extracts. Chemistry in Australia 73(8), 15.

Urban, S., Separovic, F., 2005. Developments in hyphenated spectroscopic methods in natural product profiling. Frontiers in Drug Design & Discovery 1(1), 113-166.

Vorobiof, D. A., Abratt, R., 2007. The cancer burden in Africa. South African Medical Journal 97(10), 937.

Wieler, S., Gagne, J. -P., Vaziri, H., Poirier, G. G., Benchimol, S., 2003. Poly (ADP-ribose) polymerase-1 is a positive regulator of the p53-mediated G1 arrest response following ionizing radiation. Journal of Biological Chemistry 278(21), 18914-18921.

Williams, G. H., Stoeber, K., 2012. The cell cycle and cancer. The Journal of pathology 226(2), 352-364.

Wolff, S., Erster, S., Palacios, G., Moll, U. M., 2008. p53′s mitochondrial translocation and MOMP action is independent of Puma and Bax and severely disrupts mitochondrial membrane integrity. Cell research 18(7), 733-744.

Wu, S. -Y., Pan, S. -L., Xiao, Z. -Y., Hsu, J. -L., Chen, M. -C., Lee, K. -H., Teng, C. -M., 2014. NPRL-Z-1, as a new topoisomerase II poison, induces cell apoptosis and ROS generation in human renal carcinoma cells. PloS one 9(11), e 112220.

Yin, Y., Chen, W., Tang, C., Ding, H., Jang, J., Weng, M., Cai, Y., Zou, G., 2011. NF-κB, JNK and p53 pathways are involved in tubeimoside-1-induced apoptosis in HepG2 cells with oxidative stress and G 2/M cell cycle arrest. Food and Chemical Toxicology 49(12), 3046-3054.

Yoon, M. -A., Jeong, T. -S., Park, D. -S., Xu, M. -Z., Oh, H. -W., Song, K. -B., Lee, W. S., Park, H. -Y., 2006. Antioxidant effects of quinoline alkaloids and 2,4-di-tert-butylphenol isolated from Scolopendra subspinipes. Biological and Pharmaceutical Bulletin 29(4), 735-739.

Young, D. W., Bender, A., Hoyt, J., McWhinnie, E., Chirn, G. -W., Tao, C. Y., Tallarico, J. A., Labow, M., Jenkins, J. L., Mitchison, T. J., 2008. Integrating high-content screening and ligand-target prediction to identify mechanism of action. Nature chemical biology 4(1), 59-68.

Yurdakok, B., Baydan, E., Okur, H., Gurcan, I. S., 2014. Cytotoxic effects of etephon and maleic hydrazide in Vero, Hep2, HepG2 cells. Drug and chemical toxicology 37(4), 459-465.

Zhang, F., Dossey, A. T., Zachariah, C., Edison, A. S., Brüschweiler, R., 2007. Strategy for automated analysis of dynamic metabolic mixtures by NMR. Application to an insect venom. Analytical chemistry 79(20), 7748-7752.

Zon, G., Robbins, J. D., 1983. 31 P-and 13 CN. MR-spectral and chemical characterization of the end-group and repeating-unit components of oligosaccharides derived by acid hydrolysis of haemophilus influenzae type b capsular polysaccharide. Carbohydrate research 114(1), 103-121.

Jha, B., et al., Quorum sensing inhibition by Asparagopsis taxiformis, a marine macro alga: separation of the compound that interrupts bacterial communication. Marine drugs, 2013. 11(1): p. 253-265.

### REFERENCES (SECOND SET)

1. Vorobiof D A, Abratt R. The cancer burden in Africa. South African Medical Journal. 2007; 97(10):937. PMID: 18000571

2. Cancer IAfRo. World Cancer Report 2014. Geneva: WHO. 2014.

3. Tavakoli J, Miar S, Zadehzare MM, Akbari H. Evaluation of Effectiveness of Herbal Medication in Cancer Care: A Review Study. Iranian journal of cancer preyention. 2012; 5(3):144. PMID: 25628834

4. Kuete V, Efferth T. Pharmacogenomics of Cameroonian traditional herbal medicine for cancer therapy. Journal of Ethnopharmacology. 2011; 137(1):752±66. doi.org/10.1016/j.jep.2011.06.035 PMID: 21763411

5. Giuliano K A, DeBiasio R L, Dunlay R T, Gough A, Volosky J M, Zock J, et al. High-content screening: a new approach to easing key bottlenecks in the drug discovery process. Journal of Biomolecular Screening. 1997; 2(4):249±59.

6. Taylor D L. Past, present, and future of high content screening and the field of cellomics. High Content Screening: Springer; 2006. p. 3±18.

7. Korn K, Krausz E. Cell-based high-content screening of small-molecule libraries. Current opinion in chemical biology. 2007; 11(5):503±10. doi.org/10.1016/j.cbpa.2007.08.030 PMID: 17931958

8. Haney S A, LaPan P, Pan J, Zhang J. High-content screening moves to the front of the line. Drug Discovery Today. 2006; 11(19):889±94.

9. Nichols A. High content screening as a screening tool in drug discovery. High Content Screening: Springer; 2006. p. 379±87.

10. Ghosh R N, Grove L, Lapets O. A quantitative cell-based high-content screening assay for the epidermal growth factor receptor-specific activation of mitogen-activated protein kinase. Assay and Drug Development Rechnologies. 2004; 2(5):473±81. doi.org/10.1089/adt.2004.2.473 PMID: 15671645

11. Rausch O. High content cellular screening. Current opinion in chemical biology. 2006; 10(4):316±20. doi.org/10.1016/j.cbpa.2006.06.004 PMID: 16793326

12. Diercks T, Coles M, Kessler H. Applications of NMR in drug discovery. Current Opinion in Chemical Biology. 2001; 5(3):285±91. PMID: 11479120

13. Ge Q, Zhang A Q, Sun P L. Isolation, Purification and Structural Characterization of a Novel Water-Soluble Glucan from the Fruiting Bodies of Phellinus BAUMII PILÂT. Journal of Food Biochemistry. 2010; 34 (6):1205±15.

14. Zon G, Robbins J D. 31 P-and 13 CN. MR-spectral and chemical characterization of the end-group and repeating-unit components of oligosaccharides derived by acid hydrolysis of haemophilus influenzae type b capsular polysaccharide. Carbohydrate Research. 1983; 114(1):103±21. PMID: 6601988

15. Zhang F, Dossey A T, Zachariah C, Edison A S, BruÈschweiler R. Strategy for automated analysis of dynamic metabolic mixtures by NMR. Application to an insect venom. Analytical chemistry. 2007; 79 (20):7748±52. doi.org/10.102/ac0711586 PMID: 17822309

16. Keifer PA. NMR spectroscopy in drug discovery: tools for combinatorial chemistry, natural products, and metabolism research. Progress in Drug Research: Springer; 2000. p. 137±211.

17. Al-Talla Z, Akrawi S, Emwas A. Solid state NMR and bioequivalence comparison of the pharmacokinetic parameters of two formulations of clindamycin. International Journal of Clinical Pharmacology and Therapeutics. 2011; 49(7):469±76. PMID: 21726499

18. Khorshid F, Emwas A -H, Mahboub F. The Cytotoxic Effect of Small and Large Molecules of PMF Fraction Extracted from Camel Urine on Cancer Cells. British Journal of Medicine and Medical Research. 2015.

19. Emwas A -H M, Al-Talla Z A, Yang Y, Kharbatia N M. Gas Chromatography±Mass Spectrometry of Biofluids and Extracts. Metabonomics: Methods and Protocols. 2015:91±112.

20. Emwas A -H M, Al-Talla Z A, Kharbatia N M. Sample Collection and Preparation of Biofluids and Extracts for Gas Chromatography±Mass Spectrometry. Metabonomics: Methods and Protocols. 2015:75±90.

21. Urban S. LC-NMR: profiling and dereplication of natural product extracts. Chemistry in Australia. 2006; 73(8):15.

22. Urban S, Separovic F. Developments in hyphenated spectroscopic methods in natural product profiling. Frontiers in Drug Design & Discovery. 2005; 1(1):113±66.

23. Hussain A I, Rathore H A, Sattar M Z, Chatha S A, Sarker S D, Gilani A H. Citrullus colocynthis (L.) Schrad (bitter apple fruit): A review of its phytochemistry, pharmacology, traditional uses and nutritional Anti-cancer activity of Saudi Arabian herbal plants

Hajjar, et al., PLOS One, Jun. 13, 2017, pp. 1-19, available at the website: journals.plos.org/plosone/article?id=10.1371/journal.pone.0177316

1. A composition comprising an aqueous or C₁-C₃ alcoholic extract of at least one herb selected from the group consisting of Juniperus phoenicea (Arar), Anastatica hierochuntica (Kaff Maryam) and Citrullus colocynthis (Hanzal).

2. The composition according to claim 1 wherein said extract is prepared by exposing at least one of said herbs to an effective amount of boiling water.

3. The composition according to claim 1 wherein said extract is an aqueous extract in liquid form comprising at least about 50% by volume water.

4. The composition according to claim 1 wherein said extract is an ethanolic extract in liquid form comprising at least about 50% by volume ethanol.

5. The composition according to claim 1 wherein said extract is in semi-liquid form or in solid form.

6. (canceled)

7. The composition according to claim 1, used as a food supplement, an adjuvant or therapeutic agent.

8. (canceled)

9. (canceled)

10. (canceled)

11. The composition according to claim 1 wherein said extract is an extract of two herbs selected from the group consisting of Juniperus phoenicea (Arar), Anastatica hierochuntica (Kaff Maryam) and Citrullus colocynthis (Hanzal).

12. The composition according to claim 1 wherein said extract is an extract of the herbs Juniperus phoenicea (Arar), Anastatica hierochuntica (Kaff Maryam) and Citrullus colocynthis (Hanzal).

13. The composition according to claim 1 comprising 2,2-dimethoxybutane, 2,6-dimethylbenzaldehyde, 3-trifluoroacetoxydodecane, 2,4-ditertbutylphenol, Ethyl 4-ethoxybenzoate, dodecyl acrylate, Stearic acid, hexanedioic acid, bis(2-ethylhexyl) ester, propanoic acid 3,3′-thiobis-,didodecyl ester or a mixture thereof.

14. The composition according to claim 1 comprising 2,2-dimethoxybutane, 2,6-dimethylbenzaldehyde, 3-trifluoroacetoxydodecane, 2,4-ditertbutylphenol, Ethyl 4-ethoxybenzoate, dodecyl acrylate, Stearic acid, hexanedioic acid, bis(2-ethylhexyl) ester and propanoic acid 3,3′-thiobis-,didodecyl ester.

15. The composition according to claim 1 comprising ethephon, (+)-eudesmin or burseran, sphinganine, palmitic amide, acesulfame-Na, methyl6-O[2,3,4-tris-O-(2,2-dimethylpropanoyl)-6-methyl-β-D-glucopyranuronosyl]-βDgalactopyranoside triacetate, estra-1,3,5(10)-triene-3,6beta,17beta-triol triacetate or a mixture thereof.

16. The composition according to claim 1 comprising ethephon, (+)-eudesmin or burseran, sphinganine, palmitic amide, acesulfame-Na, methyl6-O-[2,3,4-tris-O-(2,2-dimethylpropanoyl)-6-methyl-β-D-glucopyranuronosyl]-βDgalactopyranoside triacetate, and estra-1,3,5(10)-triene-3,6beta,17beta-triol triacetate.

17. A pharmaceutical composition for use in treating cancer in a patient in need comprising an anti-cancer effective amount of 2,2-dimiethoxybutane, methyl6-O-[2,3,4-tris-O-(2,2-dimethylpropanoyl)-6-methyl-β-D-glucopyranuronosyl]-βDgalactopyranoside triacetate, estra-1,3,5(10)-triene-3,6beta,17beta-triol triacetate or a mixture thereof in combination with a pharmaceutically acceptable carrier, additive or excipient.

18. A composition comprising an extract according to claim 1 in combination with a pharmaceutically acceptable carrier, additive or excipient.

19. The composition according to claim 1 further comprising an anticancer agent.

20. (canceled)

21. A method of treating, inhibiting, preventing, reducing the incidence of, ameliorating or resolving a cancerous disease state or condition in a patient in need, comprising administering to said patient a composition according to claim 1 in effective amounts.

22. A method for inhibiting topoisomerase in a patient, comprising administering to a patient a composition according to claim 1 in effective amounts.

23. A method for testing potential effectiveness of medicinal herbal compositions in treating, inhibiting, preventing, reducing the incident of, ameliorating or resolving a cancerous disease state or condition, the method comprising:
(a) selecting one or more herbs suspected of possible anticancer activity; generating fractions of said one or more herbs; using high-content screening, measuring cytological profiles of said one or more herbs; comparing said cytological profiles with a set of reference compounds with known modes of action; and executing cluster analyses of the cytological profiles to determine modes of action of compounds in the generated fractions as topoisomerase inhibitors, or,
(b) using automated microscopy in conjunction with image analysis to perform phenotypic profiling, including a characterizing of cells imaged by fluorescence cytology via high-content screening and comparing results with a standard, wherein results which are greater than or lower than the standard is an indication that the medicinal herbal composition is potentially effective against said cancerous disease state or condition.

24. The method according to claim 23 wherein said herbal compositions are aqueous or C₁-C₃ alcoholic extracts of one or more of Juniperus phoenicea (Arar), Anastatica hierochuntica (Kaff Maryam) and Citrullus colocynthis (Hanzal).

25. (canceled)
